(19) United States
(12) Patent Application Publication
(10) Pub. No.: US 2026/0193315 A1
(43) Pub. Date: Jul. 9, 2026

(54) ENGINEERED 4-1BBL VARIANTS AND METHODS OF USE THEREOF

(71) Applicant: FBD BIOLOGICS LIMITED, Kowloon (HK)

(72) Inventors: Chun-Yu Lin, Taipei City (TW); Yi-Chun Hsieh, Tainan City (TW); Shih-Han Huang, New Taipei City (TW); Chi-Ling Tseng, Taipei City (TW)

(21) Appl. No.: 19/124,540

(22) PCT Filed: Oct. 27, 2023
(86) PCT No.: PCT/CN2023/127253

§ 371 Date: Apr. 25, 2025

Related U.S. Application Data
(60) Provisional application No. 63/420,400, filed on Oct. 28, 2022.

(51) Int. Cl. C07K 14/705; A61K 38/00; A61P 35/00; C07K 19/00; C12N 5/071; C12N 5/10; C12N 15/62; C12N 15/85

(52) U.S. Cl. CPC C07K 14/70575; A61K 38/00; A61P 35/00; C07K 19/00; C12N 5/0682; C12N 5/10; C12N 15/62; C12N 15/85; C07K 2317/524; C07K 2317/526; C07K 2319/30; C12N 2510/04

(57) ABSTRACT

This disclosure provides engineered 4-1BBL variants, and methods of use thereof.

### CROSS-REFERENCE TO RELATED APPLICATION

This disclosure claims priority to and benefit of U.S. Provisional Patent Application Ser. No. 63/420,400, filed Oct. 28, 2022, which is incorporated herein by reference in its entirety.

### SEQUENCE LISTING

This application contains a Sequence Listing that has been submitted electronically as an XML file named 52246-0010WO1_ST26_SL.XML. The XML file, created on Oct. 24, 2023, is 112,677 bytes in size. The material in the XML file is hereby incorporated by reference in its entirety.

### TECHNICAL FIELD

This disclosure relates to engineered 4-1BBL variants, and methods of use thereof.

### BACKGROUND

The immune system can differentiate between normal cells in the body and those it sees as “foreign,” which allows the immune system to attack the foreign cells while leaving the normal cells alone. This mechanism sometimes involves proteins called immune checkpoints. Immune checkpoints are molecules in the immune system that either turn up a signal (co-stimulatory molecules) or turn down a signal.

The interaction between 4-1BB and 4-1BBL provide costimulatory signals to a variety of T cells, which can be used to discover cancer immunotherapy. The 4-1BB/4-1BBL complex together with a signal provided by a T-cell receptor can provide costimulatory signals to CD4+ and CD8+ T cells in mice, leading to the activation of CD4+ and CD8+ T cells. The activation of CD8+ T cells is essential in antitumor immunity. Thus, targeting 4-1BB/4-1BBL can be useful for cancer immunotherapy. However, some anti-4-1BBL antibodies (e.g., Urelumab) can induce hepatotoxicity. Thus, there is a need to develop cancer therapies targeting 4-1BB/4-1BBL pathway with limited toxicities.

### SUMMARY

This disclosure relates to engineered 4-1BBL variants, protein complexes, and methods of use thereof. The variants or protein complexes can be used to target the 4-1BB/4-1BBL pathway. The results indicate that some variants and protein complexes can effectively bind to 4-1BB-expressing cells (e.g., T cells) and induce 4-1BB-mediated downstream signaling pathway activities (e.g., NFκB activity). In addition, the protein complexes can stimulate 4-1BB-expressing immune cell (e.g., T cell) activation, proliferation, and cytokine release. In particular, the protein complexes do not overly induce cytokine release that may lead to hepatotoxicity, which has been observed using anti-4-1BB antibodies Urelumab.

Therefore, the protein complexes described herein can be used for cancer treatment with similar or higher 4-1BB binding affinity, and similar or reduced agonistic ability to activate 4-1BB/4-1BBL signaling pathway. In some embodiments, the protein complexes have enhanced T cell binding capability than wild-type 4-1BBL.

In one aspect, the disclosure is related to an engineered 4-1BBL polypeptide comprising an amino acid sequence that is at least 80% identical to SEQ ID NO: 2 or SEQ ID NO: 59, in some embodiments, the engineered 4-1BBL polypeptide comprises one or more amino acid mutations at AA′ loop, CD loop, and/or GH loop. In some embodiments, the amino acid that corresponds to S62 of SEQ ID NO: 2 is E, T, P, A, N, T, or H. In some embodiments, the engineered 4-1BBL polypeptide described herein comprises one or more of the following: (a) the amino acid that corresponds to G106 of SEQ ID NO: 2 is Q, K, H, R, F, or S; and (b) the amino acid that corresponds to E107 of SEQ ID NO: 2 is T, Q, A, R, L, M, S, or I. In some embodiments, the engineered 4-1BBL polypeptide described herein comprises one or more of the following: (a) the amino acid that corresponds to A176 of SEQ ID NO: 2 is S or Q; (b) the amino acid that corresponds to W177 of SEQ ID NO: 2 is L, M, or F; (c) the amino acid that corresponds to L179 of SEQ ID NO: 2 is F, A, or M; (d) the amino acid that corresponds to T180 of SEQ ID NO: 2 is R, S, A, or E; and (e) the amino acid that corresponds to A183 of SEQ ID NO: 2 is Q, R, or K. In some embodiments, the engineered 4-1BBL polypeptide described herein comprises one or more of the following: (a) the amino acid that corresponds to W60 of SEQ ID NO: 2 is F; and (b) the amino acid that corresponds to P64 of SEQ ID NO: 2 is N. In some embodiments, the amino acid that corresponds to L100 of SEQ ID NO: 2 is V. In some embodiments, the engineered 4-1BBL polypeptide described herein further comprises one or more of the following: (a) the amino acid that corresponds to S18 of SEQ ID NO: 2 is I; and (b) the amino acid that corresponds to L98 of SEQ ID NO: 2 is V. In some embodiments, the engineered 4-1BBL polypeptide described herein comprises one or more of the following: (a) the amino acid that corresponds to position 61 of SEQ ID NO: 2 is Y; (b) the amino acid that corresponds to position 63 of SEQ ID NO: 2 is D; (c) the amino acid that corresponds to position 65 of SEQ ID NO: 2 is G; the amino acid that corresponds to position 66 of SEQ ID NO: 2 is L; (e) the amino (d) acid that corresponds to position 101 of SEQ ID NO: 2 is R; (f) the amino acid that corresponds to position 102 of SEQ ID NO: 2 is R; (g) the amino acid that corresponds to position 103 of SEQ ID NO: 2 is V; (h) the amino acid that corresponds to position 104 of SEQ ID NO: 2 is V; (i) the amino acid that corresponds to position 105 of SEQ ID NO: 2 is A; (j) the amino acid that corresponds to position 178 of SEQ ID NO: 2 is Q; and (k) the amino acid that corresponds to position 181 of SEQ ID NO: 2 is Q. In some embodiments, the engineered 4-1BBL polypeptide described herein comprises an amino acid sequence that is at least 85%, 90%, 95%, or 100% identical to SEQ ID NO: 2, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39. In some embodiments, the engineered 4-1BBL polypeptide described herein comprises an amino acid sequence that is at least 85%, 90%, 95%, or 100% identical to SEQ ID NO: 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, or 93.

In one aspect, the disclosure is related to an engineered 4-1BBL polypeptide comprising an amino acid sequence that is at least 80% identical to SEQ ID NO: 2, 6, 59, or 60, in some embodiments, the polypeptide comprises one or more of the following: (a) the amino acid that corresponds to S62 of SEQ ID NO: 2 is E; (b) the amino acid that corresponds to E107 of SEQ ID NO: 2 is T; and (c) the amino acid that corresponds to W177 of SEQ ID NO: 2 is L. In some embodiments, the engineered 4-1BBL polypeptide described herein comprises an amino acid sequence that is at least 90% identical to SEQ ID NO: 6 or 60.

In one aspect, the disclosure is related to an engineered 4-1BBL polypeptide comprising an amino acid sequence that is at least 80% identical to SEQ ID NO: 2, 7, 59, or 61, in some embodiments, the amino acid that corresponds to T180 of SEQ ID NO: 2 is R. In some embodiments, the engineered 4-1BBL polypeptide described herein comprises an amino acid sequence that is at least 90% identical to SEQ ID NO: 7 or 61.

In one aspect, the disclosure is related to an engineered 4-1BBL polypeptide comprising an amino acid sequence that is at least 80% identical to SEQ ID NO: 2, 8, 59, or 62, in some embodiments, the polypeptide comprises one or more of the following: (a) the amino acid that corresponds to S62 of SEQ ID NO: 2 is T; and (b) the amino acid that corresponds to T180 of SEQ ID NO: 2 is S. In some embodiments, the engineered 4-1BBL polypeptide described herein comprises an amino acid sequence that is at least 90% identical to SEQ ID NO: 8 or 62.

In one aspect, the disclosure is related to an engineered 4-1BBL polypeptide comprising an amino acid sequence that is at least 80% identical to SEQ ID NO: 2, 9, 59, or 63, in some embodiments, the amino acid that corresponds to G106 of SEQ ID NO: 2 is Q. In some embodiments, the engineered 4-1BBL polypeptide described herein comprises an amino acid sequence that is at least 90% identical to SEQ ID NO: 9 or 63.

In one aspect, the disclosure is related to an engineered 4-1BBL polypeptide comprising an amino acid sequence that is at least 80% identical to SEQ ID NO: 2, 10, 59, or 64, in some embodiments, the amino acid that corresponds to T180 of SEQ ID NO: 2 is A. In some embodiments, the engineered 4-1BBL polypeptide described herein comprises an amino acid sequence that is at least 90% identical to SEQ ID NO: 10 or 64.

In one aspect, the disclosure is related to an engineered 4-1BBL polypeptide comprising an amino acid sequence that is at least 80% identical to SEQ ID NO: 2, 11, 59, or 65, in some embodiments, the amino acid that corresponds to T180 of SEQ ID NO: 2 is E. In some embodiments, the engineered 4-1BBL polypeptide described herein comprises an amino acid sequence that is at least 90% identical to SEQ ID NO: 11 or 65.

In one aspect, the disclosure is related to an engineered 4-1BBL polypeptide comprising an amino acid sequence that is at least 80% identical to SEQ ID NO: 2, 12, 59, or 66, in some embodiments, the polypeptide comprises one or more of the following: (a) the amino acid that corresponds to G106 of SEQ ID NO: 2 is K; and (b) the amino acid that corresponds to A183 of SEQ ID NO: 2 is Q. In some embodiments, the engineered 4-1BBL polypeptide described herein comprises an amino acid sequence that is at least 90% identical to SEQ ID NO: 12 or 66.

In one aspect, the disclosure is related to an engineered 4-1BBL polypeptide comprising an amino acid sequence that is at least 80% identical to SEQ ID NO: 2, 13, 59, or 67, in some embodiments, the amino acid that corresponds to S62 of SEQ ID NO: 2 is P. In some embodiments, the engineered 4-1BBL polypeptide described herein comprises an amino acid sequence that is at least 90% identical to SEQ ID NO: 13 or 67.

In one aspect, the disclosure is related to an engineered 4-1BBL polypeptide comprising an amino acid sequence that is at least 80% identical to SEQ ID NO: 2, 14, 59, or 68, in some embodiments, the polypeptide comprises one or more of the following: (a) the amino acid that corresponds to E107 of SEQ ID NO: 2 is Q; and (b) the amino acid that corresponds to A183 of SEQ ID NO: 2 is R. In some embodiments, the engineered 4-1BBL polypeptide described herein comprises an amino acid sequence that is at least 90% identical to SEQ ID NO: 14 or 68.

In one aspect, the disclosure is related to an engineered 4-1BBL polypeptide comprising an amino acid sequence that is at least 80% identical to SEQ ID NO: 2, 15, 59, or 69, in some embodiments, the amino acid that corresponds to L98 of SEQ ID NO: 2 is V. In some embodiments, the engineered 4-1BBL polypeptide described herein comprises an amino acid sequence that is at least 90% identical to SEQ ID NO: 15 or 69.

In one aspect, the disclosure is related to an engineered 4-1BBL polypeptide comprising an amino acid sequence that is at least 80% identical to SEQ ID NO: 2, 16, 59, or 70, in some embodiments, the polypeptide comprises one or more of the following: (a) the amino acid that corresponds to G106 of SEQ ID NO: 2 is H; and (b) the amino acid that corresponds to T180 of SEQ ID NO: 2 is A. In some embodiments, the engineered 4-1BBL polypeptide described herein comprises an amino acid sequence that is at least 90% identical to SEQ ID NO: 16 or 70.

In one aspect, the disclosure is related to an engineered 4-1BBL polypeptide comprising an amino acid sequence that is at least 80% identical to SEQ ID NO: 2, 17, 59, or 71, in some embodiments, the amino acid that corresponds to E107 of SEQ ID NO: 2 is A. In some embodiments, the engineered 4-1BBL polypeptide described herein comprises an amino acid sequence that is at least 90% identical to SEQ ID NO: 17 or 71.

In one aspect, the disclosure is related to an engineered 4-1BBL polypeptide comprising an amino acid sequence that is at least 80% identical to SEQ ID NO: 2, 18, 59, or 72, in some embodiments, the polypeptide comprises one or more of the following: (a) the amino acid that corresponds to S62 of SEQ ID NO: 2 is A; and (b) the amino acid that corresponds to E107 of SEQ ID NO: 2 is Q. In some embodiments, the engineered 4-1BBL polypeptide described herein comprises an amino acid sequence that is at least 90% identical to SEQ ID NO: 18 or 72.

In one aspect, the disclosure is related to an engineered 4-1BBL polypeptide comprising an amino acid sequence that is at least 80% identical to SEQ ID NO: 2, 19, 59, or 73, in some embodiments, the polypeptide comprises one or more of the following: (a) the amino acid that corresponds to S62 of SEQ ID NO: 2 is N; and (b) the amino acid that corresponds to E107 of SEQ ID NO: 2 is Q. In some embodiments, the engineered 4-1BBL polypeptide described herein comprises an amino acid sequence that is at least 90% identical to SEQ ID NO: 19 or 73.

In one aspect, the disclosure is related to an engineered 4-1BBL polypeptide comprising an amino acid sequence that is at least 80% identical to SEQ ID NO: 2, 20, 59, or 74, in some embodiments, the polypeptide comprises one or more of the following: (a) the amino acid that corresponds to S62 of SEQ ID NO: 2 is T; and (b) the amino acid that corresponds to A183 of SEQ ID NO: 2 is R. In some embodiments, the engineered 4-1BBL polypeptide described herein comprises an amino acid sequence that is at least 90% identical to SEQ ID NO: 20 or 74.

In one aspect, the disclosure is related to an engineered 4-1BBL polypeptide comprising an amino acid sequence that is at least 80% identical to SEQ ID NO: 2, 21, 59, or 75, in some embodiments, the amino acid that corresponds to S62 of SEQ ID NO: 2 is H. In some embodiments, the engineered 4-1BBL polypeptide described herein comprises an amino acid sequence that is at least 90% identical to SEQ ID NO: 21 or 75.

In one aspect, the disclosure is related to an engineered 4-1BBL polypeptide comprising an amino acid sequence that is at least 80% identical to SEQ ID NO: 2, 22, 59, or 76, in some embodiments, the polypeptide comprises one or more of the following: (a) the amino acid that corresponds to S62 of SEQ ID NO: 2 is P; (b) the amino acid that corresponds to E107 of SEQ ID NO: 2 is R; and (c) the amino acid that corresponds to L179 of SEQ ID NO: 2 is F. In some embodiments, the engineered 4-1BBL polypeptide described herein comprises an amino acid sequence that is at least 90% identical to SEQ ID NO: 22 or 76.

In one aspect, the disclosure is related to an engineered 4-1BBL polypeptide comprising an amino acid sequence that is at least 80% identical to SEQ ID NO: 2, 23, 59, or 77, in some embodiments, the polypeptide comprises one or more of the following: (a) the amino acid that corresponds to E107 of SEQ ID NO: 2 is L; and (b) the amino acid that corresponds to W177 of SEQ ID NO: 2 is M. In some embodiments, the engineered 4-1BBL polypeptide described herein comprises an amino acid sequence that is at least 90% identical to SEQ ID NO: 23 or 77.

In one aspect, the disclosure is related to an engineered 4-1BBL polypeptide comprising an amino acid sequence that is at least 80% identical to SEQ ID NO: 2, 24, 59, or 78, in some embodiments, the polypeptide comprises one or more of the following: (a) the amino acid that corresponds to S62 of SEQ ID NO: 2 is P; (b) the amino acid that corresponds to G106 of SEQ ID NO: 2 is R; and (c) the amino acid that corresponds to W177 of SEQ ID NO: 2 is M. In some embodiments, the engineered 4-1BBL polypeptide described herein comprises an amino acid sequence that is at least 90% identical to SEQ ID NO: 24 or 78.

In one aspect, the disclosure is related to an engineered 4-1BBL polypeptide comprising an amino acid sequence that is at least 80% identical to SEQ ID NO: 2, 25, 59, or 79, in some embodiments, the amino acid that corresponds to S18 of SEQ ID NO: 2 is I. In some embodiments, the engineered 4-1BBL polypeptide described herein comprises an amino acid sequence that is at least 90% identical to SEQ ID NO: 25 or 79.

In one aspect, the disclosure is related to an engineered 4-1BBL polypeptide comprising an amino acid sequence that is at least 80% identical to SEQ ID NO: 2, 26, 59, or 80, in some embodiments, the amino acid that corresponds to L179 of SEQ ID NO: 2 is A. In some embodiments, the engineered 4-1BBL polypeptide described herein comprises an amino acid sequence that is at least 90% identical to SEQ ID NO: 26 or 80.

In one aspect, the disclosure is related to an engineered 4-1BBL polypeptide comprising an amino acid sequence that is at least 80% identical to SEQ ID NO: 2, 27, 59, or 81, in some embodiments, the polypeptide comprises one or more of the following: (a) the amino acid that corresponds to S62 of SEQ ID NO: 2 is A; and (b) the amino acid that corresponds to L100 of SEQ ID NO: 2 is V. In some embodiments, the engineered 4-1BBL polypeptide described herein comprises an amino acid sequence that is at least 90% identical to SEQ ID NO: 27 or 81.

In one aspect, the disclosure is related to an engineered 4-1BBL polypeptide comprising an amino acid sequence that is at least 80% identical to SEQ ID NO: 2, 28, 59, or 82, in some embodiments, the polypeptide comprises one or more of the following: (a) the amino acid that corresponds to W60 of SEQ ID NO: 2 is F; (b) the amino acid that corresponds to E107 of SEQ ID NO: 2 is T; and (c) the amino acid that corresponds to L179 of SEQ ID NO: 2 is M. In some embodiments, the engineered 4-1BBL polypeptide described herein comprises an amino acid sequence that is at least 90% identical to SEQ ID NO: 28 or 82.

In one aspect, the disclosure is related to an engineered 4-1BBL polypeptide comprising an amino acid sequence that is at least 80% identical to SEQ ID NO: 2, 29, 59, or 83, in some embodiments, the amino acid that corresponds to E107 of SEQ ID NO: 2 is Q. In some embodiments, the engineered 4-1BBL polypeptide described herein comprises an amino acid sequence that is at least 90% identical to SEQ ID NO: 29 or 83.

In one aspect, the disclosure is related to an engineered 4-1BBL polypeptide comprising an amino acid sequence that is at least 80% identical to SEQ ID NO: 2, 30, 59, or 84, in some embodiments, the amino acid that corresponds to A183 of SEQ ID NO: 2 is K. In some embodiments, the engineered 4-1BBL polypeptide described herein comprises an amino acid sequence that is at least 90% identical to SEQ ID NO: 30 or 84.

In one aspect, the disclosure is related to an engineered 4-1BBL polypeptide comprising an amino acid sequence that is at least 80% identical to SEQ ID NO: 2, 31, 59, or 85, in some embodiments, the polypeptide comprises one or more of the following: (a) the amino acid that corresponds to W60 of SEQ ID NO: 2 is F; and (b) the amino acid that corresponds to G106 of SEQ ID NO: 2 is R. In some embodiments, the engineered 4-1BBL polypeptide described herein comprises an amino acid sequence that is at least 90% identical to SEQ ID NO: 31 or 85.

In one aspect, the disclosure is related to an engineered 4-1BBL polypeptide comprising an amino acid sequence that is at least 80% identical to SEQ ID NO: 2, 32, 59, or 86, in some embodiments, the polypeptide comprises one or more of the following: (a) the amino acid that corresponds to S62 of SEQ ID NO: 2 is T; (b) the amino acid that corresponds to E107 of SEQ ID NO: 2 is M; and (c) the amino acid that corresponds to A176 of SEQ ID NO: 2 is S. In some embodiments, the engineered 4-1BBL polypeptide described herein comprises an amino acid sequence that is at least 90% identical to SEQ ID NO: 32 or 86.

In one aspect, the disclosure is related to an engineered 4-1BBL polypeptide comprising an amino acid sequence that is at least 80% identical to SEQ ID NO: 2, 33, 59, or 87, in some embodiments, the polypeptide comprises one or more of the following: (a) the amino acid that corresponds to S62 of SEQ ID NO: 2 is A; and (b) the amino acid that corresponds to E107 of SEQ ID NO: 2 is S. In some embodiments, the engineered 4-1BBL polypeptide described herein comprises an amino acid sequence that is at least 90% identical to SEQ ID NO: 33 or 87.

In one aspect, the disclosure is related to an engineered 4-1BBL polypeptide comprising an amino acid sequence that is at least 80% identical to SEQ ID NO: 2, 34, 59, or 88, in some embodiments, the amino acid that corresponds to A176 of SEQ ID NO: 2 is Q. In some embodiments, the engineered 4-1BBL polypeptide described herein comprises an amino acid sequence that is at least 90% identical to SEQ ID NO: 34 or 88.

In one aspect, the disclosure is related to an engineered 4-1BBL polypeptide comprising an amino acid sequence that is at least 80% identical to SEQ ID NO: 2, 35, 59, or 89, in some embodiments, the polypeptide comprises one or more of the following: (a) the amino acid that corresponds to S62 of SEQ ID NO: 2 is P; (b) the amino acid that corresponds to G106 of SEQ ID NO: 2 is F; and (c) the amino acid that corresponds to A176 of SEQ ID NO: 2 is S. In some embodiments, the engineered 4-1BBL polypeptide described herein comprises an amino acid sequence that is at least 90% identical to SEQ ID NO: 35 or 89.

In one aspect, the disclosure is related to an engineered 4-1BBL polypeptide comprising an amino acid sequence that is at least 80% identical to SEQ ID NO: 2, 36, 59, or 90, in some embodiments, the amino acid that corresponds to E107 of SEQ ID NO: 2 is S. In some embodiments, the engineered 4-1BBL polypeptide described herein comprises an amino acid sequence that is at least 90% identical to SEQ ID NO: 36 or 90.

In one aspect, the disclosure is related to an engineered 4-1BBL polypeptide comprising an amino acid sequence that is at least 80% identical to SEQ ID NO: 2, 37, 59, or 91, in some embodiments, the polypeptide comprises one or more of the following: (a) the amino acid that corresponds to G106 of SEQ ID NO: 2 is S; and (b) the amino acid that corresponds to W177 of SEQ ID NO: 2 is F. In some embodiments, the engineered 4-1BBL polypeptide described herein comprises an amino acid sequence that is at least 90% identical to SEQ ID NO: 37 or 91.

In one aspect, the disclosure is related to an engineered 4-1BBL polypeptide comprising an amino acid sequence that is at least 80% identical to SEQ ID NO: 2, 38, 59, or 92, in some embodiments, the polypeptide comprises one or more of the following: (a) the amino acid that corresponds to P64 of SEQ ID NO: 2 is N; and (b) the amino acid that corresponds to E107 of SEQ ID NO: 2 is I. In some embodiments, the engineered 4-1BBL polypeptide described herein comprises an amino acid sequence that is at least 90% identical to SEQ ID NO: 38 or 92.

In one aspect, the disclosure is related to an engineered 4-1BBL polypeptide comprising an amino acid sequence that is at least 80% identical to SEQ ID NO: 2, 39, 59, or 93, in some embodiments, the polypeptide comprises one or more of the following: (a) the amino acid that corresponds to S62 of SEQ ID NO: 2 is P; and (b) the amino acid that corresponds to E107 of SEQ ID NO: 2 is A. In some embodiments, the engineered 4-1BBL polypeptide described herein comprises an amino acid sequence that is at least 90% identical to SEQ ID NO: 39 or 93.

In some embodiments, the engineered 4-1BBL polypeptide further comprises a CH2 domain and a CH3 domain. In some embodiments, the engineered 4-1BBL polypeptide further comprises a hinge region. In some embodiments, the CH2 domain is an IgG CH2 domain and the CH3 domain is an IgG CH3 domain. In some embodiments, the engineered 4-1BBL polypeptide comprises an amino acid sequence that is at least 80%, 85%, 90%, or 95% identical to any one of SEQ ID NOs: 41-58.

In one aspect, the disclosure is related to a protein construct comprising the engineered 4-1BBL polypeptide described herein. In some embodiments, the protein construct described herein comprises two or more engineered 4-1BBL polypeptides. In some embodiments, at least two engineered 4-1BBL polypeptides are identical. In some embodiments, at least two engineered 4-1BBL polypeptides are different. In some embodiments, the protein construct described herein further comprises an Fc region. In some embodiments, the Fc region is an IgG4 Fc region. In some embodiments, the Fc region is an IgG1 Fc region (e.g., with LALA mutations or LALA-PG mutations). In some embodiments, the engineered 4-1BBL polypeptide is connected to the C-terminus of the Fc region, optionally via a linker peptide. In some embodiments, the engineered 4-1BBL polypeptide is connected to the N-terminus of the Fc region, optionally via a linker peptide.

In one aspect, the disclosure is related to a protein construct comprising a first fusion polypeptide comprising the engineered 4-1BBL polypeptide described herein, a first CH2 domain, and a first CH3 domain; and a second fusion polypeptide comprising a second CH2 domain, and a second CH3 domain. In some embodiments, the first fusion polypeptide and the second fusion polypeptide associate with each other, forming a dimer. In some embodiments, the second fusion polypeptide further comprises a second engineered 4-1BBL polypeptide.

In one aspect, the disclosure is related to a pharmaceutical composition comprising the engineered 4-1BBL polypeptide or the protein construct described herein; and a pharmaceutically acceptable carrier.

In one aspect, the disclosure is related to nucleic acid encoding the engineered 4-1BBL polypeptide or the protein construct described herein.

In one aspect, the disclosure is related to a vector comprising the nucleic acid described herein. In one aspect, the disclosure is related to a cell comprising the nucleic acid described herein. In some embodiments, the cell is a CHO cell.

In one aspect, the disclosure is related to a method of producing an engineered 4-1BBL polypeptide or a protein construct comprising the engineered 4-1BBL polypeptide, the method comprising (a) culturing the cell described herein under conditions sufficient for the cell to produce the engineered 4-1BBL polypeptide or the protein construct; and (b) collecting the engineered 4-1BBL polypeptide or the protein construct produced by the cell.

In one aspect, the disclosure is related to a method of treating a subject having cancer, the method comprising administering a therapeutically effective amount of a composition comprising the engineered 4-1BBL polypeptide or the protein construct described herein, to the subject. In some embodiments, the subject has a solid tumor or a hematologic cancer. In some embodiments, the cancer is breast cancer, oropharyngeal cancer, ovarian cancer, B cell lymphoma, or Non-Hodgkin's lymphoma, non-small cell lung cancer (NSCLC), melanoma, B-cell non-Hodgkin lymphoma, colorectal cancer, or multiple myeloma.

In one aspect, the disclosure is related to a method of decreasing the rate of tumor growth, the method comprising contacting a tumor cell with an effective amount of a composition comprising the engineered 4-1BBL polypeptide or the protein construct described herein.

In one aspect, the disclosure is related to a method of killing a tumor cell, the method comprising contacting a tumor cell with an effective amount of a composition comprising the engineered 4-1BBL polypeptide or the protein construct described herein.

As used herein, the term “engineered 4-1BBL polypeptide” refers to a polypeptide derived from a wild-type 4-1BBL polypeptide or a portion thereof (e.g., the extracellular region of 4-1BBL), optionally with one or more mutations (e.g., insertions, deletions, or substitutions). In some embodiments, the engineered 4-1BBL polypeptide comprises or consists of the extracellular region of 4-1BBL or variants thereof. In some embodiments, the engineered 4-1BBL polypeptide comprises or consists of the TNF homology domain (THD) of 4-1BBL or variants thereof. In some embodiments, the engineered 4-1BBL polypeptide is a modified THD domain. In some embodiments, the 4-1BBL extracellular region has one or more mutations. In some embodiments, the THD domain has one or more mutations.

As used herein, the term “protein construct” refers to a complex having one or more polypeptides. In some embodiments, the protein construct has two or more polypeptides, wherein the polypeptides can associate with each other, forming a dimer or a multimer (e.g., a trimer).

As used herein, the term “cancer” refers to cells having the capacity for uncontrolled autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows the amino acid residues of human 4-1BBL extracellular domain. Residues at the binding interface between 4-1BBL and its receptor 4-1BB are shaded. Amino acid positions in SEQ ID NO: 1 are labeled on top of selected residues.

FIG. 2A shows the screening results of 1st round of selected 4-1BBL variant clones.

FIG. 2B shows the screening results of 2nd round of selected 4-1BBL variant clones.

FIG. 2C summarizes 4-1BBL phagemid screening results.

FIG. 3 shows a table summarizing the mutated residues of selected 4-1BBL variants.

FIG. 4A shows the screening results of 34 unique 4-1BBL variants.

FIG. 4B summarizes the characteristics of 34 unique 4-1BBL variants on 4-1BB binding activity and 4-1BB-mediated NF-κB reporter activity.

FIG. 5 shows the HPLC-SEC analysis results of Fc fused 4-1BBL variants.

FIGS. 6A-6B show the binding activity of G4Fc-4-1BBL variants on activated T cells.

FIGS. 6C-6D show the activity of G4Fc-4-1BBL variants on induction of 4-1BB-mediated NF-κB reporter activity.

FIG. 6E summarizes the characteristics of the selected G4Fc-4-1BBL variants on 4-1BB binding activity and 4-1BB-mediated NF-κB reporter activity.

FIG. 7A shows the effects of selected G4Fc-4-1BBL variants on T cell proliferation. SIRPα-G4Fc-wt (Trillium; SEQ ID NO: 4) was used as a negative control. PF05082566 and BMS663513 were used as positive controls.

FIG. 7B shows the effects of selected G4Fc-4-1BBL variants on IFN-γ production. SIRPα-G4Fc-wt (Trillium) was used as a negative control. PF05082566 and BMS663513 were used as positive controls.

FIG. 8A shows the effects of selected G4Fc-4-1BBL variants on T cell proliferation. SIRPα-G4Fc-wt (Trillium) was used as a negative control. PF05082566 and BMS663513 were used as positive controls.

FIG. 8B shows the effects of selected G4Fc-4-1BBL variants on IFN-γ production. SIRPα-G4Fc-wt (Trillium) was used as a negative control. PF05082566 and BMS663513 were used as positive controls.

FIG. 8C shows the effects of selected G4Fc-4-1BBL variants on IL-2 production. SIRPα-G4Fc-wt (Trillium) was used as a negative control. PF05082566 and BMS663513 were used as positive controls.

FIG. 9A shows the binding activity of selected G4Fc-4-1BBL variants on activated T cells. An anti-Her2 antibody (HLX22, developed by Henlix) was used as a negative control.

FIG. 9B shows the activity of selected G4Fc-4-1BBL variants on induction of 4-1BB-mediated NF-κB reporter activity. SIRPα-G4Fc-wt (Trillium) was used as a negative control.

FIG. 10A shows the effects of selected G4Fc-4-1BBL variants on T cell proliferation. SIRPα-G4Fc-wt (Trillium) was used as a negative control.

FIG. 10B shows the effects of selected G4Fc-4-1BBL variants on IL2 production. SIRPα-G4Fc-wt (Trillium) was used as a negative control.

FIG. 10C shows the effects of selected G4Fc-4-1BBL variants on IFN-γ production. SIRPα-G4Fc-wt (Trillium) was used as a negative control.

FIG. 11A shows the effects of selected G4Fc-4-1BBL variants on IL-27 production. Urelumab (BMS663513) was used as positive controls. Utomliumab (PF05082566) and SIRPα-G4Fc-wt (Trillium) were used as a negative control.

FIG. 11B shows the effects of selected G4Fc-4-1BBL variants on TNF-α production. Urelumab (BMS663513) and Utomliumab (PF05082566) were used as positive controls. SIRPα-G4Fc-wt (Trillium) was used as a negative control.

FIG. 12 lists amino acid sequences of the wild-type 4-1BBL extracellular domain and variants thereof. The sequences correspond to amino acids 50-254 of human 4-1BBL (SEQ ID NO: 1).

FIG. 13 lists amino acid sequences of the wild-type 4-1BBL TNF homology domain and variants thereof. The sequences correspond to amino acids 90-241 of human 4-1BBL (SEQ ID NO: 1).

FIG. 14 lists protein sequences discussed in the disclosure.

### DETAILED DESCRIPTION

TNFSF9 (also known as 4-1BB ligand, 4-1BBL, CD137L, tumor necrosis factor ligand superfamily member 9 (TNFSF9)) is a type II transmembrane protein of the TNF superfamily primarily on antigen-presenting cells, such as IFN-γ activated macrophages, CD40 ligand activated B cells, monocytes, T cells, dendritic cells (DC), and B cells. TNFSF9 on the cell membrane can transmit a reverse signal, thereby inhibiting the proliferation of activated T cells and inducing their apoptosis. The reverse signal can also induce monocyte activation, promote the secretion of IL-6, IL-8 and TNF-Ade, and prolong cell survival. In addition, the reverse signal can stimulate the maturation of DC derived from CD34+ hematopoietic stem cells. Northern blot analysis revealed multiple TNFSF9 transcripts in brain, placenta, lung, skeletal muscle, and kidney, as well as in activated T cells, transformed B cells, and monocyte lines.

The membranous form of 4-1BBL exists as a trimer, and upon engagement with its receptor on T cells, it delivers a robust costimulatory signal. 4-1BBL was found to be expressed following stimulation on professional APCs including DCs and macrophages as well as activated B cells in both human and mice. Human 4-1BBL message was detected as early as 30 minutes following stimulation through immobilized CD3 monoclonal antibody (mAb) and peaks at 1 hour. 4-1BBL was also present at high levels in the sera of some patients with hematological diseases 35 as well as on some carcinoma cell lines.

A detailed description of TNFSF9 and its function can be found, e.g., in Cheuk, Adam TC, et al., “Role of 4-1BB: 4-1BB ligand in cancer immunotherapy.” Cancer Gene Therapy 11.3 (2004): 215-226; and Li, Yan, et al., “Limited cross-linking of 4-1BB by 4-1BB ligand and the agonist monoclonal antibody Utomilumab.” Cell Reports 25.4 (2018): 909-920; each of which is incorporated by reference in its entirety.

The present disclosure provides engineered 4-1BBL variants. These engineered 4-1BBL variants can be used to target 4-1BB/4-1BBL pathway, whereas the interaction of engineered 4-1BBL variants and 4-1BB is carefully modulated.

### Engineered 4-1BBL Variants

4-1BBL belongs to the TNF superfamily and is expressed on the surface of antigen presenting cells, including e.g., dendritic cells, B cells, and macrophages. The synergistic stimulatory signal produced by the interaction of 4-1BB and its receptor 4-1BBL induces activation and proliferation of T cells and NK cells, and the production of cytokines. Human 4-1BBL includes, from N-terminus to C-terminus, a cytoplasmic region, a transmembrane region, and an extracellular region. According to the UniProt Database (UniProt ID: P41273), the cytoplasmic region of human 4-1BBL corresponds to amino acids 1-28 of SEQ ID NO: 1, the transmembrane region of human 4-1BBL corresponds to amino acids 29-49 of SEQ ID NO: 1, and the extracellular region of human 4-1BBL corresponds to amino acids 50-254 of SEQ ID NO: 1. The extracellular region of human 4-1BB includes a TNF homology domain (or THD domain) that corresponds to amino acids 90-241 of SEQ ID NO: 1. In some embodiments, sequence of the THD domain (also referred to as the 4-1BBLv2 sequence) is shown in SEQ ID NO: 59. In some embodiments, the sequence corresponding to amino acids 64-254 of SEQ ID NO: 1 is also referred to as the 4-1BBLv1 sequence (SEQ ID NO: 94).

The 4-1BB/4-1BBL complex consists of three monomeric 4-1BBs bound to a trimeric 4-1BBL. Each 4-1BB monomer binds to two 4-1BBLs via cysteine-rich domains (CRDs). The interaction between 4-1BB and the second 4-1BBL is required to stabilize their interactions. The link with 4-1BBL is largely made up of amino acids from the dynamic loops of the CRD2 and the β sheet of CRD3 of 4-1BB, according to a detailed study of the binding between the 4-1BB and 4-1BBL interface. CRD2 amino acids (T61, Q67, and K69) interact with the AA′ loop (e.g., Y110 and G114) and the GH loop (e.g., Q227 and Q230) of 4-1BBL to form various hydrogen bond interactions. Details can be found, e.g., in Li, Y., et al. “Limited cross-linking of 4-1BB by 4-1BB ligand and the agonist monoclonal antibody Utomilumab.” Cell Reports 25.4 (2018): 909-920, which is incorporated herein by reference in its entirety.

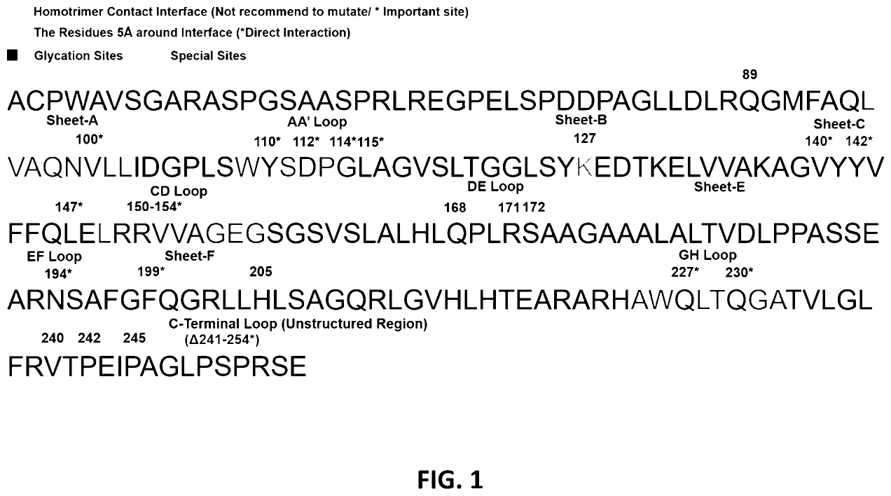

Based on the structure of human 4-1BB in complex with human 4-1BBL, the binding interface between 4-1BBL and 4-1BB can be determined. Residues that are located within about 5 Å of the binding interface are selected for mutagenesis analysis, e.g., by constructing a phagemid library to generate random mutations. As shown in FIG. 1, multiple interacting residues are located within AA′ loop (corresponding to amino acids 109-117 of SEQ ID NO: 1, or amino acids 60-68 of SEQ ID NO: 2), CD loop (corresponding to amino acids 149-157 of SEQ ID NO: 1, or amino acids 100-108 of SEQ ID NO: 2), and GH loop (corresponding to amino acids 225-232 of SEQ ID NO: 1, or amino acids 176-183 of SEQ ID NO: 2). These regions are the targets for mutations. Thus, in some embodiments, the engineered 4-1BBL variant (e.g., any of the engineered 4-1BBL polypeptides described herein) comprises or consists of one or more amino acid mutations at AA′ loop, CD loop, and/or GH loop.

In addition, the present disclosure shows that Val100, Tyr110, Asp112, Glyl14, Leu115, Val140, Tyr142, Leu147, Arg150, Arg151, Val152, Val153, Ala154, Asn194, Phe199, Gln227, and Gln230 in SEQ ID NO: 1 can be important for maintaining the homotrimer contact interface of human 4-1BBL. Thus, in some embodiments, these amino acid residues are retained.

In summary, Ser67, Trp109, Ser111, Pro113, Leu147, Leu149, Glyl55, Glu156, Ala225, Trp226, Leu228, Thr229, and Ala232 in SEQ ID NO: 1 are identified as candidate amino acids for human 4-1BBL mutation screening. These residues correspond to Ser18, Trp60, Ser62, Pro64, Leu98, Leu100, Glyl06, Glu107, Ala176, Trp177, Leu179, Thr180, and Ala183 respectively, in SEQ ID NO: 2.

Thus, in one aspect, the engineered 4-1BBL polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 2, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, or 93. In some embodiments, the engineered 4-1BBL polypeptide described herein comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1 or SEQ ID NO: 94, wherein the amino acid sequence comprises one or more of the mutations described herein.

In some embodiments, the engineered 4-1BBL polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 7 or 61. In some embodiments, the engineered 4-1BBL polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 9 or 63. In some embodiments, the engineered 4-1BBL polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 16 or 70. In some embodiments, the engineered 4-1BBL polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 18 or 72. In some embodiments, the engineered 4-1BBL polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 23 or 77. In some embodiments, the engineered 4-1BBL polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 26 or 80. In some embodiments, the engineered 4-1BBL polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 27 or 81.

In some embodiments, the engineered 4-1BBL variants can have at least or about 1 (e.g., at least or about 2, 3, 4, 5, 6, 7, 8, 9, 10, 20, 30, 40) amino acid insertions, deletions, or substitutions as compared to any one of SEQ ID NOs: 2, 6-39, and 59-93.

In some embodiments, The engineered 4-1BBL polypeptide comprises or consists of one or more of the following mutations:
(a) the amino acid that corresponds to S62 of SEQ ID NO: 2 is E, T, P, A, N, T, or H;
(b) the amino acid that corresponds to G106 of SEQ ID NO: 2 is Q, K, H, R, F, or S; and
(c) the amino acid that corresponds to E107 of SEQ ID NO: 2 is T, Q, A, R, L, M, S, or I.

In some embodiments, the engineered 4-1BBL polypeptide comprises or consists of one or more of the following mutations:
(a) the amino acid that corresponds to A176 of SEQ ID NO: 2 is S or Q;
(b) the amino acid that corresponds to W177 of SEQ ID NO: 2 is L, M, or F;
(c) the amino acid that corresponds to L179 of SEQ ID NO: 2 is F, A, or M;
(d) the amino acid that corresponds to T180 of SEQ ID NO: 2 is R, S, A, or E; and
(e) the amino acid that corresponds to A183 of SEQ ID NO: 2 is Q, R, or K.

In some embodiments, the engineered 4-1BBL polypeptide comprises or consists of one or more of the following mutations:
(a) the amino acid that corresponds to W60 of SEQ ID NO: 2 is F; and
(b) the amino acid that corresponds to P64 of SEQ ID NO: 2 is N.

In some embodiments, the amino acid that corresponds to L100 of SEQ ID NO: 2 is V.

In some embodiments, the engineered 4-1BBL polypeptide comprises or consists of one or more of the following mutations:
(a) the amino acid that corresponds to S18 of SEQ ID NO: 2 is I; and
(b) the amino acid that corresponds to L98 of SEQ ID NO: 2 is V.

In some embodiments, the engineered 4-1BBL polypeptide comprises or consists of one or more of the following residues:
(a) the amino acid that corresponds to position 61 of SEQ ID NO: 2 is Y;
(b) the amino acid that corresponds to position 63 of SEQ ID NO: 2 is D;
(c) the amino acid that corresponds to position 65 of SEQ ID NO: 2 is G;
(d) the amino acid that corresponds to position 66 of SEQ ID NO: 2 is L;
(e) the amino acid that corresponds to position 101 of SEQ ID NO: 2 is R;
(f) the amino acid that corresponds to position 102 of SEQ ID NO: 2 is R;
(g) the amino acid that corresponds to position 103 of SEQ ID NO: 2 is V;
(h) the amino acid that corresponds to position 104 of SEQ ID NO: 2 is V;
(i) the amino acid that corresponds to position 105 of SEQ ID NO: 2 is A;
(j) the amino acid that corresponds to position 178 of SEQ ID NO: 2 is Q; and
(k) the amino acid that corresponds to position 181 of SEQ ID NO: 2 is Q.

In some embodiments, the engineered 4-1BBL polypeptide comprises or consists of one or more of the following mutations:
(a) the amino acid that corresponds to S18 of SEQ ID NO: 2 is I;
(b) the amino acid that corresponds to W60 of SEQ ID NO: 2 is F;
(c) the amino acid that corresponds to S62 of SEQ ID NO: 2 is E, T, P, A, N, T, or H;
(d) the amino acid that corresponds to P64 of SEQ ID NO: 2 is N;
(e) the amino acid that corresponds to L98 of SEQ ID NO: 2 is V;
(f) the amino acid that corresponds to L100 of SEQ ID NO: 2 is V;
(g) the amino acid that corresponds to G106 of SEQ ID NO: 2 is Q, K, H, R, F, or S;
(h) the amino acid that corresponds to E107 of SEQ ID NO: 2 is T, Q, A, R, L, M, S, or I;
(i) the amino acid that corresponds to A176 of SEQ ID NO: 2 is S or Q;
(j) the amino acid that corresponds to W177 of SEQ ID NO: 2 is L, M, or F;
(k) the amino acid that corresponds to L179 of SEQ ID NO: 2 is F, A, or M;
(l) the amino acid that corresponds to T180 of SEQ ID NO: 2 is R, S, A, or E; and
(m) the amino acid that corresponds to A183 of SEQ ID NO: 2 is Q, R, or K.

In some embodiments, the engineered 4-1BBL polypeptide comprises or consists of one or more of the following mutations:
(a) the amino acid that corresponds to S62 of SEQ ID NO: 2 is E;
(b) the amino acid that corresponds to E107 of SEQ ID NO: 2 is T; and
(c) the amino acid that corresponds to W177 of SEQ ID NO: 2 is L.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 6, 59, or 60).

In some embodiments, the engineered 4-1BBL polypeptide comprises or consists of an amino acid sequence, in which the amino acid that corresponds to T180 of SEQ ID NO: 2 is R. In some embodiments, the engineered 4-1BBL polypeptide comprises or consists of an amino acid sequence that is at least 80%, 85%, 90%, or 95% identical to any one of SEQ ID NOs: 2, and 2, 6-39, and 59-93 (e.g., SEQ ID NO: 2, 7, 59, or 61).

In some embodiments, the engineered 4-1BBL polypeptide comprises or consists of one or more of the following:
(a) the amino acid that corresponds to S62 of SEQ ID NO: 2 is T; and
(b) the amino acid that corresponds to T180 of SEQ ID NO: 2 is S.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 8, 59, or 62).

In some embodiments, the engineered 4-1BBL polypeptide comprises or consists of an amino acid sequence, in which the amino acid that corresponds to G106 of SEQ ID NO: 2 is Q. In some embodiments, the engineered 4-1BBL polypeptide comprises or consists of an amino acid sequence that is at least 80%, 85%, 90%, or 95% identical to any one of SEQ ID NOs: 2, and 2, 6-39, and 59-93 (e.g., SEQ ID NO: 2, 9, 59, or 63).

In some embodiments, the engineered 4-1BBL polypeptide comprises or consists of an amino acid sequence, in which the amino acid that corresponds to T180 of SEQ ID NO: 2 is A. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 10, 59, or 64).

In some embodiments, the engineered 4-1BBL polypeptide comprises or consists of an amino acid sequence, in which the amino acid that corresponds to T180 of SEQ ID NO: 2 is E. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 11, 59, or 65).

In some embodiments, the engineered 4-1BBL polypeptide comprises or consists of one or more of the following:
(a) the amino acid that corresponds to G106 of SEQ ID NO: 2 is K; and
(b) the amino acid that corresponds to A183 of SEQ ID NO: 2 is Q.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 12, 59, or 66).

In some embodiments, the engineered 4-1BBL polypeptide comprises or consists of an amino acid sequence, in which the amino acid that corresponds to S62 of SEQ ID NO: 2 is P. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 13, 59, or 67).

In some embodiments, the engineered 4-1BBL polypeptide comprises or consists of one or more of the following:
(a) the amino acid that corresponds to E107 of SEQ ID NO: 2 is Q; and
(b) the amino acid that corresponds to A183 of SEQ ID NO: 2 is R.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 14, 59, or 68).

In some embodiments, the engineered 4-1BBL polypeptide comprises or consists of an amino acid sequence, in which the amino acid that corresponds to L98 of SEQ ID NO: 2 is V. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 15, 59, or 69).

In some embodiments, the engineered 4-1BBL polypeptide comprises or consists of one or more of the following:
(a) the amino acid that corresponds to G106 of SEQ ID NO: 2 is H; and
(b) the amino acid that corresponds to T180 of SEQ ID NO: 2 is A.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 16, 59, or 70).

In some embodiments, the engineered 4-1BBL polypeptide comprises or consists of an amino acid sequence, in which the amino acid that corresponds to E107 of SEQ ID NO: 2 is A. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 17, 59, or 71).

In some embodiments, the engineered 4-1BBL polypeptide comprises or consists of one or more of the following mutations:
(a) the amino acid that corresponds to S62 of SEQ ID NO: 2 is A; and
(b) the amino acid that corresponds to E107 of SEQ ID NO: 2 is Q.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 18, 59, or 72).

In some embodiments, the engineered 4-1BBL polypeptide comprises or consists of one or more of the following mutations:
(a) the amino acid that corresponds to S62 of SEQ ID NO: 2 is N; and
(b) the amino acid that corresponds to E107 of SEQ ID NO: 2 is Q.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 19, 59, or 73).

In some embodiments, the engineered 4-1BBL polypeptide comprises or consists of one or more of the following:
(a) the amino acid that corresponds to S62 of SEQ ID NO: 2 is T; and
(b) the amino acid that corresponds to A183 of SEQ ID NO: 2 is R.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 20, 59, or 74).

In some embodiments, the engineered 4-1BBL polypeptide comprises or consists of an amino acid sequence, in which the amino acid that corresponds to S62 of SEQ ID NO: 2 is H. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 21, 59, or 75).

In some embodiments, the engineered 4-1BBL polypeptide comprises or consists of one or more of the following mutations:
(a) the amino acid that corresponds to S62 of SEQ ID NO: 2 is P;
(b) the amino acid that corresponds to E107 of SEQ ID NO: 2 is R; and
(c) the amino acid that corresponds to L179 of SEQ ID NO: 2 is F.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 22, 59, or 76).

In some embodiments, the engineered 4-1BBL polypeptide comprises or consists of one or more of the following mutations:
(a) the amino acid that corresponds to E107 of SEQ ID NO: 2 is L; and
(b) the amino acid that corresponds to W177 of SEQ ID NO: 2 is M.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 23, 59, or 77).

In some embodiments, the engineered 4-1BBL polypeptide comprises or consists of one or more of the following mutations:
(a) the amino acid that corresponds to S62 of SEQ ID NO: 2 is P;
(b) the amino acid that corresponds to G106 of SEQ ID NO: 2 is R; and
(c) the amino acid that corresponds to W177 of SEQ ID NO: 2 is M.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 24, 59, or 78).

In some embodiments, the engineered 4-1BBL polypeptide comprises or consists of an amino acid sequence, in which the amino acid that corresponds to S18 of SEQ ID NO: 2 is I. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 25, 59, or 79).

In some embodiments, the engineered 4-1BBL polypeptide comprises or consists of an amino acid sequence, in which the amino acid that corresponds to L179 of SEQ ID NO: 2 is A. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 26, 59, or 80).

In some embodiments, the engineered 4-1BBL polypeptide comprises or consists of one or more of the following mutations:
(a) the amino acid that corresponds to S62 of SEQ ID NO: 2 is A; and
(b) the amino acid that corresponds to L100 of SEQ ID NO: 2 is V.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 27, 59, or 81).

In some embodiments, the engineered 4-1BBL polypeptide comprises or consists of one or more of the following mutations:
(a) the amino acid that corresponds to W60 of SEQ ID NO: 2 is F;
(b) the amino acid that corresponds to E107 of SEQ ID NO: 2 is T; and
(c) the amino acid that corresponds to L179 of SEQ ID NO: 2 is M.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 28, 59, or 82).

In some embodiments, the engineered 4-1BBL polypeptide comprises or consists of an amino acid sequence, in which the amino acid that corresponds to E107 of SEQ ID NO: 2 is Q. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 29, 59, or 83).

In some embodiments, the engineered 4-1BBL polypeptide comprises or consists of an amino acid sequence, in which the amino acid that corresponds to A183 of SEQ ID NO: 2 is K. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 30, 59, or 84).

In some embodiments, the engineered 4-1BBL polypeptide comprises or consists of one or more of the following mutations:
(a) the amino acid that corresponds to W60 of SEQ ID NO: 2 is F; and
(b) the amino acid that corresponds to G106 of SEQ ID NO: 2 is R.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 31, 59, or 85).

In some embodiments, the engineered 4-1BBL polypeptide comprises or consists of one or more of the following mutations:
(a) the amino acid that corresponds to S62 of SEQ ID NO: 2 is T;
(b) the amino acid that corresponds to E107 of SEQ ID NO: 2 is M; and
(c) the amino acid that corresponds to A176 of SEQ ID NO: 2 is S.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 32, 59, or 86).

In some embodiments, the engineered 4-1BBL polypeptide comprises or consists of one or more of the following mutations:
(a) the amino acid that corresponds to S62 of SEQ ID NO: 2 is A; and
(b) the amino acid that corresponds to E107 of SEQ ID NO: 2 is S.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 33, 59, or 87).

In some embodiments, the engineered 4-1BBL polypeptide comprises or consists of an amino acid sequence, in which the amino acid that corresponds to A176 of SEQ ID NO: 2 is Q. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 34, 59, or 88).

In some embodiments, the engineered 4-1BBL polypeptide comprises or consists of one or more of the following mutations:
(a) the amino acid that corresponds to S62 of SEQ ID NO: 2 is P;
(b) the amino acid that corresponds to G106 of SEQ ID NO: 2 is F; and
(c) the amino acid that corresponds to A176 of SEQ ID NO: 2 is S.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 35, 59, or 89).

In some embodiments, the engineered 4-1BBL polypeptide comprises or consists of an amino acid sequence, in which the amino acid that corresponds to E107 of SEQ ID NO: 2 is S. In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 36, 59, or 90).

In some embodiments, the engineered 4-1BBL polypeptide comprises or consists of one or more of the following mutations:
(a) the amino acid that corresponds to G106 of SEQ ID NO: 2 is S; and
(b) the amino acid that corresponds to W177 of SEQ ID NO: 2 is F.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 37, 59, or 91).

In some embodiments, the engineered 4-1BBL polypeptide comprises or consists of one or more of the following mutations:
(a) the amino acid that corresponds to P64 of SEQ ID NO: 2 is N; and
(b) the amino acid that corresponds to E107 of SEQ ID NO: 2 is I.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 38, 59, or 92).

In some embodiments, the engineered 4-1BBL polypeptide comprises or consists of one or more of the following mutations:
(a) the amino acid that corresponds to S62 of SEQ ID NO: 2 is P; and
(b) the amino acid that corresponds to E107 of SEQ ID NO: 2 is A.

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 2, 39, 59, or 93).

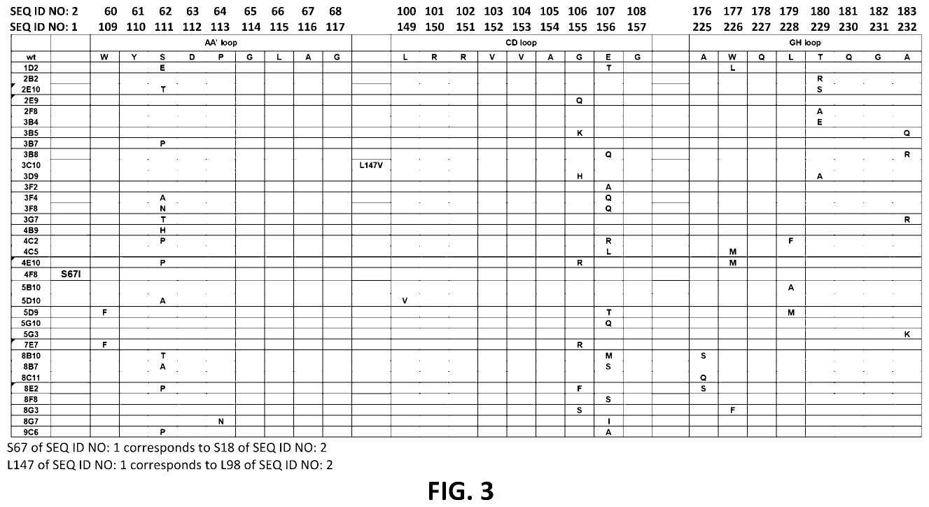

In some embodiments, the engineered 4-1BBL polypeptide comprises or consists of an amino acid sequence that is at least 80%, 85%, 90%, or 95% identical to any one of SEQ ID NOs: 2, 6-39, and 59-93 (e.g., SEQ ID NO: 7, SEQ ID NO: 16, SEQ ID NO: 61, or SEQ ID NO: 70) with 1, 2, 3, 4, 5, 6, 7, 8, 9, 10, 11, 12, 13, 14, or 15 mutations as shown in FIG. 3.

The engineered 4-1BBL polypeptide can have additional modifications. In some embodiments, the engineered 4-1BBL polypeptide can have a CH2 domain and/or a CH3 domain of Fc. In some embodiments, the engineered 4-1BBL polypeptide can be linked to the N-terminus of the CH2 domain (e.g., through an optional hinge region or a GS linker). In some embodiments, the engineered 4-1BBL polypeptide can be linked to the C-terminus of the CH3 domain (e.g., through an optional GS linker). In some embodiments, the hinge region is an IgG hinge region (e.g., IgG4 hinge region). In some embodiments, the CH2 domain is an IgG CH2 domain (e.g., IgG4 CH2 domain). In some embodiments, the CH3 domain is an IgG CH3 domain (e.g., IgG4 CH3 domain). In some embodiments, the hinge region, the CH2 domain, the CH3 domain have a sequence that is at least 80%, 85%, 90%, 95%, 100% identical to SEQ ID NO: 40.

In some embodiments, the engineered 4-1BBL polypeptides described herein can also include a tag (e.g., His tag) to facilitate screening and/or detection. In some embodiments, the tag has a sequence that is at least 80%, 85%, 90%, 95%, 96%, 97%, 98%, 99%, or 100% identical to any one of SEQ ID NOs: 95-98. In some embodiments, the tag is connected to the N-terminus or C-terminus of any engineered 4-1BBL polypeptides described herein.

### 4-1BBL Protein Constructs

The disclosure provides engineered 4-1BBL protein constructs that can specifically bind to 4-1BB. In some embodiments, these protein constructs have a similar affinity towards 4-1BB but with reduced agonistic ability. In some embodiments, these protein constructs have a higher binding affinity towards 4-1BB but with similar agonistic ability. In some embodiments, these protein constructs have a better thermostability than the wild-type 4-1BBL.

In some embodiments, the engineered 4-1BBL protein constructs can comprise any engineered 4-1BBL variant as described herein. In some embodiments, the engineered 4-1BBL protein constructs can have a sequence that is at least 80%, 81%, 82%, 83%, 84%, 85%, 86%, 87%, 88%, 89% 90%, 91%, 92%, 93%, 94%, 95%, 96%, 97%, 98%, 99% or 100% identical to any sequence of SEQ ID NOs: 59-93. In some embodiments, the engineered 4-1BBL protein constructs can comprise or consists of a sequence that is at least 80%, 81%, 82%, 83%, 84%, 85%, 86%, 87%, 88%, 89% 90%, 91%, 92%, 93%, 94%, 95%, 96%, 97%, 98%, 99% or 100% identical to any sequence of SEQ ID NOs: 41-58.

The disclosure also provides a nucleic acid comprising a polynucleotide encoding a polypeptide comprising a sequence that is at least 80%, 81%, 82%, 83%, 84%, 85%, 86%, 87%, 88%, 89% 90%, 91%, 92%, 93%, 94%, 95%, 96%, 97%, 98%, 99% or 100% identical to any sequence of SEQ ID NOs: 2, 6-39, and 59-93; or any sequence of SEQ ID NOs: 41-58.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The engineered 4-1BBL protein constructs can further comprises an Fc region of an antibody. These antibodies can be of any type (e.g., IgG, IgE, IgM, IgD, IgA, and IgY), class or subclass (e.g., IgG1, IgG2, IgG3, IgG4, IgA1, IgA2, IgE1, IgE2). In some embodiments, the he Fc region is derived from human IgG (e.g., IgG1, IgG2, IgG3, or IgG4). In some embodiments, the Fc region is an IgG4 Fc region (e.g., human IgG4 Fc region).

In some embodiments, the engineered 4-1BBL variant is linked to the Fc region through an antibody hinge region (e.g., IgG, IgE hinge region). In addition, the Fc region can be modified to provide desired effector functions or serum half-life.

The engineered 4-1BBL variants and protein constructs described herein can block the binding between endogenous 4-1BB and endogenous 4-1BBL that are expressed on immune cells. In some embodiments, by binding to 4-1BB, the engineered 4-1BBL variants and protein constructs can inhibit the binding of 4-1BB (e.g., that is expressed on T cells) to endogenous 4-1BBL that is expressed on antigen-presenting cells (e.g., dendritic cells, macrophages and B cells). Because of the retained 4-1BB binding affinity and reduced agonistic ability, the 4-1BBL variants and protein complexes described herein can maintain the potency to induce T cell activation and proliferation, while having minimal hepatotoxicity.

In some embodiments, the engineered 4-1BBL variants and protein constructs as described herein can increase immune response, activity or number of immune cells (e.g., T cells) by at least 10%, 20%, 30%, 40%, 50%, 60%, 70%, 80%, 90%, 100%, 2 folds, 3 folds, 5 folds, 10 folds, or 20 folds.

In some implementations, the engineered 4-1BBL variants and protein constructs can bind to 4-1BB (e.g., human 4-1BB, monkey 4-1BB (e.g., cynomolgus monkey (Macaca fascicularis), mouse 4-1BB) with a dissociation rate (k_{off}) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (k_{off}) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kₒₙ) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kₒₙ) is less than 1×10⁵/Ms, less than 1×10⁶/Ms, or less than 1×10⁷/Ms.

Affinities can be deduced from the quotient of the kinetic rate constants (KD=k_{off}/kₒₙ). In some embodiments, KD is less than 1×10⁻⁶ M, less than 1×10⁻⁷M, less than 1×10⁻⁸ M, less than 1×10⁻⁹ M, or less than 1×10⁻¹⁰ M. In some embodiments, the KD is less than 300 nM, 200 nM, 100 nM, 50 nM, 30 nM, 20 nM, 15 nM, 10 nM, 9 nM, 8 nM, 7 nM, 6 nM, 5 nM, 4 nM, 3 nM, 2 nM, 1 nM, 900 μM, 800 μM, 700 μM, 600 μM, 500 μM, 400 μM, 300 μM, 200 pM, 100 μM, 90 μM, 80 μM, 70 μM, 60 μM, 50 μM, 40 μM, 30 μM, 20 μM, or 10 μM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity include, e.g., ELISA, radioimmunoassay (RIA), and surface plasmon resonance (SPR). In some embodiments, the engineered 4-1BBL variants and protein constructs can bind to monkey 4-1BB, and/or mouse 4-1BB. In some embodiments, the engineered 4-1BBL variants and protein constructs cannot bind to monkey 4-1BB, and/or mouse 4-1BB.

In some embodiments, thermal stabilities are determined. The engineered 4-1BBL variants and protein constructs as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the engineered 4-1BBL variants and/or protein constructs as described herein has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the engineered 4-1BBL variants and/or protein constructs as described herein has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI(%)=[1−(Ti−T0)/(Vi−V0)]×100

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the tumor inhibitory effects of the engineered 4-1BBL variants and/or protein constructs as described herein are comparable to an anti-4-1BB reference antibody. In some embodiments, the tumor inhibitory effect of the engineered 4-1BBL variants and/or protein constructs as described herein is at least 50%, at least 60%, at least 70%, at least 80%, at least 90%, at least 100%, at least 110%, at least 120%, at least 130%, at least 140%, at least 150%, at least 2-fold, at least 3-fold, at least 4-fold, at least 5-fold, or at least 10-fold as compared to that of an anti-4-1BB reference antibody.

In some embodiments, the protein constructs as described herein have a functional Fc region. In some embodiments, the Fc region is human IgG1, human IgG2, human IgG3, or human IgG4. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the protein constructs as described herein have an Fc region without effector function. In some embodiments, the Fc is a human IgG4 Fc. In some embodiments, the Fc does not have a functional Fc region. For example, the Fc region has LALA mutations (L234A and L235A mutations in EU numbering), or LALA-PG mutations (L234A, L235A, P329G mutations in EU numbering).

In some embodiments, the engineered 4-1BBL variant (e.g., any of the engineered 4-1BBL variants described herein) is linked to the N-terminus or C-terminus of the Fc region. In some embodiments, the engineered 4-1BBL variant is linked to the Fc region via a linker peptide. In some embodiments, the linker peptide includes a sequence that is at least 80%, 85%, 90%, 95%, or 100% identical to any one of SEQ ID NO: 99 or 100. In some embodiments, the linker peptide includes a sequence that includes 1, 2, 3, 4, 5, 6, 7, or 8 repeats of GGGGS (SEQ ID NO: 5).

In some embodiments, provided herein are protein constructs that include, from N-terminus to C-terminus, a 4-1BBL variant (e.g., any of the 4-1BBL variants described herein), a linker peptide (e.g., any of the linker peptides described herein), and a human IgG4 hinge region and Fc region (e.g., SEQ ID NO: 40). In some embodiments, the 4-1BBL variant includes a sequence that is at least at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 2, 6-39, and 59-93. In some embodiments, the 4-1BBL variant includes one or more (e.g., at least 1, at least 2, at least 3, at least 4, at least 5, at least 6, at least 7, at least 8, at least 9, or at least 10) of the mutations described herein.

Some other modifications to the Fc region can be made. For example, a cysteine residue(s) can be introduced into the Fc region, thereby allowing interchain disulfide bond formation in this region. The homodimeric fusion protein thus generated may have any increased half-life in vitro and/or in vivo.

In some embodiments, the IgG4 has S228P mutation (EU numbering). The S228P mutation prevents in vivo and in vitro IgG4 Fab-arm exchange.

In some embodiments, Fc regions are provided having a carbohydrate structure that lacks fucose attached (directly or indirectly) to an Fc region. For example, the amount of fucose in such Fc region composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues; or position 314 in Kabat numbering); however, Asn297 may also be located about ±3 amino acids upstream or downstream of position 297, i.e., between positions 294 and 300, due to minor sequence variations in Fc region sequences. Such fucosylation variants may have improved ADCC function. In some embodiments, to reduce glycan heterogeneity, the Fc region can be further engineered to replace the Asparagine at position 297 with Alanine (N297A).

In some embodiments, the binding affinity between 4-1BB (e.g., human 4-1BB, monkey 4-1BB, mouse 4-1BB, or extracellular domains thereof) and the engineered 4-1BBL variants and/or protein constructs as described herein is at least 1-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, or 50-fold as compared to that between 4-1BB and a wild-type 4-1BBL or protein constructs thereof.

In some embodiments, the engineered 4-1BBL variants and/or protein constructs as described herein has a B/E ratio (4-1BB binding OD₄₅₀ over expression OD₄₅₀) that is between 0.1-0.5, between 0.5-1, between 1 and 1.25, between 1.25 and 1.5, between 1.5 and 1.75, between 1.75 and 2, or greater than 2. In some instances, the B/E ratio is greater than 0.4. In some embodiments, the B/E ratio is determined at 25° C. or 45° C.

In some embodiments, the engineered 4-1BBL variants and/or protein constructs as described herein has a R/E ratio (Reporter assay (RLU) over expression OD₄₅₀) that is between 0.5-1, between 1 and 1.5, between 1.5 and 2, between 2 and 2.5, between 2.5 and 3, between 3 to 3.5, between 3.5 to 4, or above 4 . . . . In some instances, the R/E ratio is greater than 0.5.

In some embodiments, the main peak of HPLC-SEC accounts for at least 70%, 75%, 80%, 85%, 90%, 95%, 96%, 97%, 98%, 99%, or 99.5% of the engineered 4-1BBL variants and/or protein constructs as described herein.

In some embodiments, the engineered 4-1BBL variants and/or protein constructs thereof as described herein can bind to human 4-1BB with an affinity that is at least 1-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, or 50-fold as compared to that of a wild-type 4-1BBL or protein constructs thereof (e.g., 4-1BBL (SEQ ID NO: 1); 4-1BBL extracellular domain (SEQ ID NO: 2); 4-1BBLv2 (SEQ ID NO: 59); or 4-1BBLv1 (SEQ ID NO: 94)). In some embodiments, the engineered 4-1BBL variants and/or protein constructs thereof as described herein can bind to 4-1BB with an affinity that is at least 50%, at least 60%, at least 70%, at least 80%, at least 90%, at least 100%, at least 110%, at least 120%, at least 130%, at least 140%, or at least 150% as compared to that of a wild-type 4-1BBL or protein constructs thereof (e.g., 4-1BBL (SEQ ID NO: 1); 4-1BBL extracellular domain (SEQ ID NO: 2); 4-1BBLv2 (SEQ ID NO: 59); or 4-1BBLv1 (SEQ ID NO: 94)).

In some embodiments, the engineered 4-1BBL variants and/or protein constructs thereof as described herein can bind to human 4-1BB-expressing T cells (e.g., T cells isolated from human PBMCs) with an affinity that is at least 1-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, or 50-fold as compared to that of a wild-type 4-1BBL or protein constructs thereof (e.g., G4Fc-4-1BBLv2). In some embodiments, the engineered 4-1BBL variants and/or protein constructs thereof as described herein can bind to 4-1BB-expressing T cells (e.g., T cells isolated from human PBMCs) with an affinity that is at least 50%, at least 60%, at least 70%, at least 80%, at least 90%, at least 100%, at least 110%, at least 120%, at least 130%, at least 140%, or at least 150% as compared to that of a wild-type 4-1BBL or protein constructs thereof (e.g., G4Fc-4-1BBLv2).

In some embodiments, the engineered 4-1BBL variants and/or protein constructs thereof as described herein can induce 4-1BB-mediated NFκB activity in a comparable manner as compared to a wild-type 4-1BBL or protein constructs thereof (e.g., G4Fc-4-1BBLv2).

In some embodiments, the engineered 4-1BBL variants and/or protein constructs thereof as described herein can induce proliferation of T cells (e.g., pre-activated T cells isolated from human PBMCs) with an proliferation rate that is at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 250%, at least 300%, at least 400%, or at least 500% as compared to a wild-type 4-1BBL or protein constructs thereof (e.g., G4Fc-4-1BBLv2) or a protein construct targeting a different antigen (e.g., SIRPα-G4Fc-wt).

In some embodiments, the engineered 4-1BBL variants and/or protein constructs thereof as described herein can induce release of cytokines (e.g., IFN-γ or IL2) that is at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 250%, at least 300%, at least 400%, at least 500%, at least 600%, at least 700%, at least 800%, at least 900%, at least 1000%, at least 20-fold, at least 30-fold, at least 40-fold, at least 50-fold, at least 60-fold, at least 70-fold, at least 80-fold, at least 90-fold, at least 100-fold, at least 500-fold, at least 1000-fold, at least 5000-fold, or at least 10000-fold as compared to a wild-type 4-1BBL or protein constructs thereof (e.g., G4Fc-4-1BBLv2) or a protein construct targeting a different antigen (e.g., SIRPα-G4Fc-wt).

In some embodiments, the engineered 4-1BBL variants and/or protein constructs thereof as described herein can induce hepatotoxicity-related cytokines (e.g., IL-27 or TNF-α) that is less than 90%, less than 80%, less than 70%, less than 60%, less than 50%, less than 40%, less than 30%, less than 20%, less than 10%, less than 5%, less than 4%, less than 3%, less than 2%, or less than 1% as compared to Urelumab or Utomilumab.

In some embodiments, the engineered 4-1BBL variants and/or protein constructs thereof as described herein can inhibit tumor growth.

### Methods of Making Engineered 4-1BBL Variants and Protein Constructs

Variants of the 4-1BBL described herein can be prepared by introducing appropriate nucleotide changes into the DNA encoding a 4-1BBL peptide or a part thereof or by peptide synthesis. Such variants include, for example, deletions, insertions, or substitutions of residues within the amino acids sequences. In some embodiments, random mutations can be introduced to residues within the AA′ loop, CD loop, and/or GH loop of human 4-1BBL. In some embodiments, the random mutations are introduced by constructing a phagemid library.

Screening can be performed. In a population of such variants, some engineered 4-1BBL variants will have increased affinity for the 4-1BB. Any combination of deletions, insertions, and/or combinations can be made to arrive at a variant that has increased binding affinity for the target. The amino acid changes introduced into the variant can also alter or introduce new post-translational modifications into the polypeptide, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Engineered 4-1BBL variants can be derived from any species of animal, including mammals. Non-limiting examples of 4-1BBL variants include 4-1BBL variants derived from humans, primates, e.g., monkeys and apes, cows, pigs, horses, sheep, camelids (e.g., camels and llamas), chicken, goats, and rodents (e.g., rats, mice, hamsters and rabbits).

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. Techniques for incorporating DNA into such expression systems are well known to those of ordinary skill in the art. The DNA may also be “naked.” The uptake of naked DNA may be increased by coating the DNA onto biodegradable beads that are efficiently transported into the cells.

For expression, the DNA insert comprising a polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding a polypeptide of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., 4-1BBL variants) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The engineered 4-1BBL variants and protein constructs of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of engineered 4-1BBL variants and protein constructs disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the engineered 4-1BBL variants and protein constructs, vector comprising the polynucleotide encoding the engineered 4-1BBL variants and protein constructs, and/or compositions thereof is to be administered, a severity of symptoms and a route of administration, and thus administration can be determined on an individual basis.

An effective amount can be administered in one or more administrations. By way of example, an effective amount of the engineered 4-1BBL variants and/or protein constructs is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount may vary, depending on, inter alia, patient history as well as other factors such as the type (and/or dosage) of the engineered 4-1BBL variants and protein constructs used.

Effective amounts and schedules for administering the engineered 4-1BBL variants and protein constructs, the polynucleotides encoding the engineered 4-1BBL variants and protein constructs, and/or compositions disclosed herein may be determined empirically, and making such determinations is within the skill in the art. Those skilled in the art will understand that the dosage that must be administered will vary depending on, for example, the mammal that will receive the engineered 4-1BBL variants and protein constructs, the polynucleotides, and/or compositions disclosed herein, the route of administration, the particular type of polynucleotides, and/or compositions disclosed herein used and other drugs being administered to the mammal.

A typical daily dosage of an effective amount of the engineered 4-1BBL variants and/or protein constructs is 0.1 mg/kg to 100 mg/kg (mg per kg of patient weight). In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg. In some embodiments, the dosage is about 1 to 10 mg/kg, about 1 to 5 mg/kg, or about 2 to 5 mg/kg.

In any of the methods described herein, the engineered 4-1BBL variants and protein constructs can be administered to the subject at least once a week (e.g., once a week, twice a week, three times a week, four times a week, once a day, twice a day, or three times a day).

In some embodiments, the one or more additional therapeutic agents can be administered to the subject prior to, or after administering the engineered 4-1BBL variants and protein constructs. In some embodiments, the one or more additional therapeutic agents are administered to the subject such that there is an overlap in the bioactive period of the one or more additional therapeutic agents and the engineered 4-1BBL variants and protein constructs in the subject.

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-SIRPα antibody, an anti-CD47 antibody, an anti-LAG-3 antibody, an anti-TIGIT antibody, an anti-BTLA antibody, an anti-CTLA-4 antibody, or an anti-GITR antibody. In some embodiments, the additional therapeutic agent is an anti-CD20 antibody (e.g., rituximab) or an anti-EGF receptor antibody (e.g., cetuximab).

### Pharmaceutical Compositions and Routes of Administration

Also provided herein are pharmaceutical compositions that contain the engineered 4-1BBL variants and protein constructs described herein. The pharmaceutical compositions can be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the agents can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Compositions containing the engineered 4-1BBL variants and protein constructs described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the engineered 4-1BBL variants and protein constructs can be formulated in aqueous solutions, preferably in physiologically-compatible buffers to reduce discomfort at the site of injection. The solution can contain formulatory agents such as suspending, stabilizing and/or dispersing agents. Alternatively the engineered 4-1BBL variants and protein constructs can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Exemplary doses include milligram or microgram amounts of any of the engineered 4-1BBL variants and protein constructs described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; about 1 μg/kg to about 50 μg/kg; about 1 mg/kg to about 10 mg/kg; or about 1 mg/kg to about 5 mg/kg). While these doses cover a broad range, one of ordinary skill in the art will understand that therapeutic agents can vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the engineered 4-1BBL variants and protein constructs in vivo.

The pharmaceutical compositions can be included in a container, pack, or dispenser together with instructions for administration. The disclosure also provides methods of manufacturing the engineered 4-1BBL variants and protein constructs for various uses as described herein.

### EXAMPLES

The invention is further described in the following examples, which do not limit the scope of the invention described in the claims.

### Example 1. Design of Engineered Extracellular Domain of Human 4-1BBL

The extracellular domain of 4-1BBL is from residue 50 to 254 of a wild-type human 4-1BBL protein (SEQ ID NO: 1), which belongs to the TNF-α homology domain. Sheet-A, AA′ loop, CD loop, and GH loop are the major regions that participate in interaction with 4-1BB. As shown in FIG. 1, the residues marked by star symbols are those that directly interact with 4-1BB, and the residues marked in light gray color are those designed for random mutation.

To obtain different 4-1BBL variants with random mutations at residues that are located within 5 Å around the binding interface between 4-1BBL and 4-1BB (based on PDB Identifiers 6A3V and 2X29), a phagemid library was constructed with a 2.8×10⁷ library size. 4-1BBL variants with different characteristics (e.g., binding activity and agonist function) were selected by screening from this phagemid library. The expression of 4-1BBL variants were induced by 1 mM of IPTG. Supernatant was collected for determination of 4-1BBL variants expression and binding activity. The supernatant were further incubated at 45° C. for 30 minutes for evaluation of thermal-stability of the 4-1BBL variants.

Specifically, 20 μl of ECOM competent cells (TG1) were transformed with the phage library expressing 4-1BBL variants. The transformed cells were spread onto a LB plate (containing 150 μg/ml of ampicillin), which was then incubated at 37° C. overnight. Single colonies from the plate were used to inoculate 900 μl of 2YT medium (containing 150 μg/ml ampicillin and 10% phosphate buffer), which was incubated at 37° C. for 3-4 hours until the OD600 value of the bacterial culture reached 0.9-1. Expression of 4-1BBL variants were induced by adding 1 mM IPTG, and the culture was then incubated at 30° C. overnight.

After centrifugation, culture supernatant was collected for determination of expression and binding activity of the 4-1BBL variants. To determine expression of the 4-1BBL variants, a 96-well plate (flat bottom) was coated with 2 μg/ml anti-His tag antibody overnight. The pre-coated plate was then blocked with 5% milk in PBS at room temperature (RT) for 1 hour. After blocking, 30 μl of bacterial supernatant was added to each well of the plate, and the plate was incubated for 1 hour at RT. After the incubation, anti-c-myc-HRP was added, and the expression level of 4-1BBL variants can be determined by measuring OD450 in a plate reader. To determine binding activity of the 4-1BBL variants, a 96-well plate (flat bottom) was coated with 0.5 μg/ml of 4-1BB overnight. The pre-coated plate was then blocked with 5% milk in PBS at RT for 1 hour. After blocking, 30μ of bacterial supernatant was added to each well of the plate, and the plate was incubated for 1 hour at RT. After the incubation, anti-c-myc-HRP was added, and the 4-1BB-binding activity of 4-1BBL variants can be determined by measuring OD450 in a plate reader.

For thermal stability test, the supernatant was incubated at 45° C. for 30 minutes, and then the expression and 4-1BB-binding activity can be determined using the methods described above.

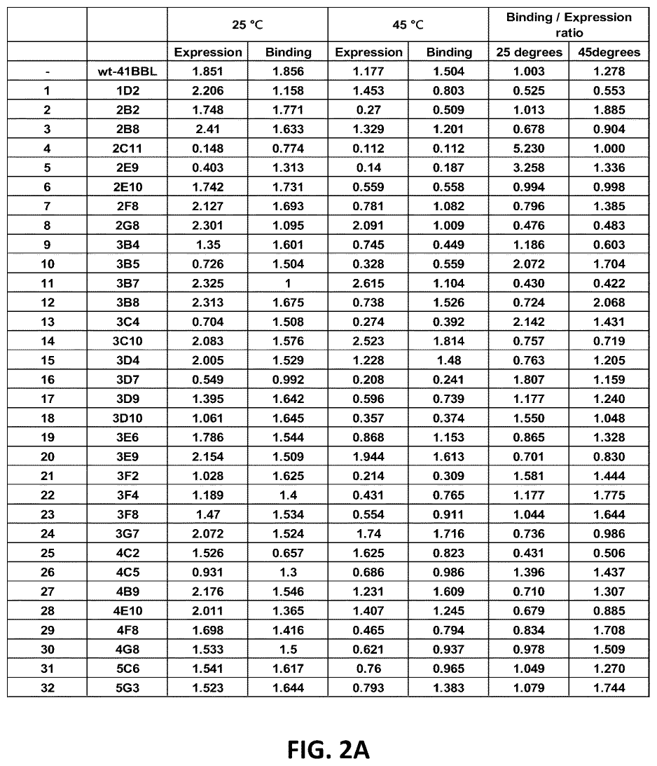

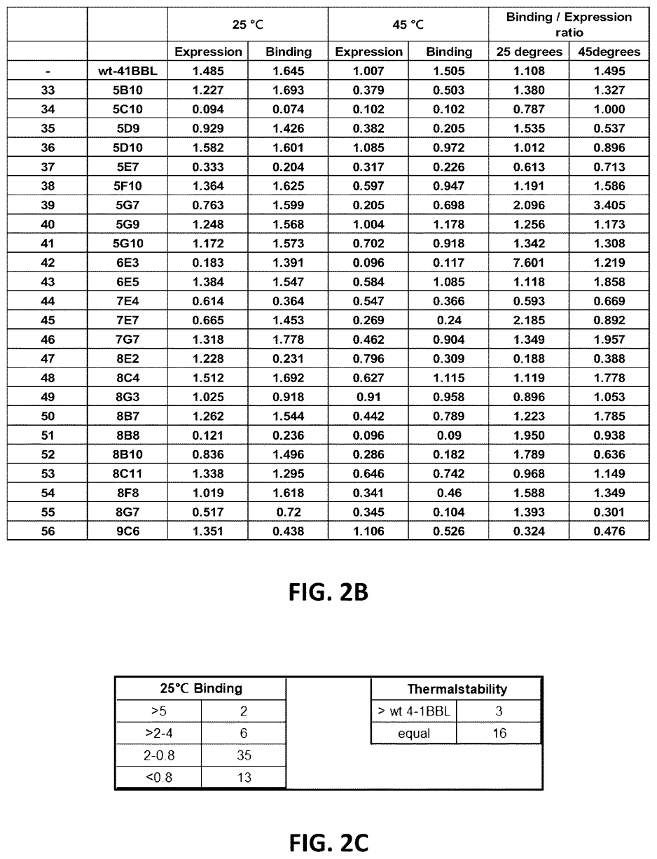

After 2 rounds of screening, 56 potential 4-1BBL variants were obtained with different characteristics (FIGS. 2A-2B). As shown in FIG. 2C, 2 clones showed 5 folds of enhancement on 4-1BB binding, and 6 clones showed 2-4 folds of enhancement on 4-1BB binding. 35 clones showed similar binding activity and 13 clones showed weaker binding activity as compared to wild-type 4-1BBL (peG4Fc-4-1BBLv2; SEQ ID NO: 3). Compared to wild-type 4-1BBL, 3 clones showed better thermal-stability and 16 clones showed similar thermal-stability.

### Example 2. Verification of 4-1BB Binding and Reporter Activities

After sequencing, 34 unique sequences were obtained from the 56 potential clones. The mutated residues are showed in FIG. 3.

The 4-1BB-binding activity and 4-1BB-mediated reporter activity of the 34 unique 4-1BBL variants were verified as follows. Briefly, the expression of the 34 unique 4-1BBL variants were induced by 1 mM of IPTG at 30° C. overnight. After centrifugation, culture supernatant was further collected for determination of 4-1BB-binding activity and 4-1BB reporter activity.

The 4-1BB-binding activity was measured using the same methods described above. With respect to 4-1BB-mediated NF-κB reporter activity, a 96-well plate (flat bottom) was coated with 5 μg/ml anti-c-myc antibody at 4° C. overnight. The induced 4-1BBL variant supernatants were added to the pre-coated plate, and the plate was incubated at 37° C. for 2 hours. The above steps were repeated for six times. Afterwards, 1×10⁵/well of transfected NF-κB Jurkat cells expressing 4-1BB was added to the plate, which was then incubated for 5 hours at 37° C. After the incubation, the luminance signal was detected to determine the 4-1BB reporter activity.

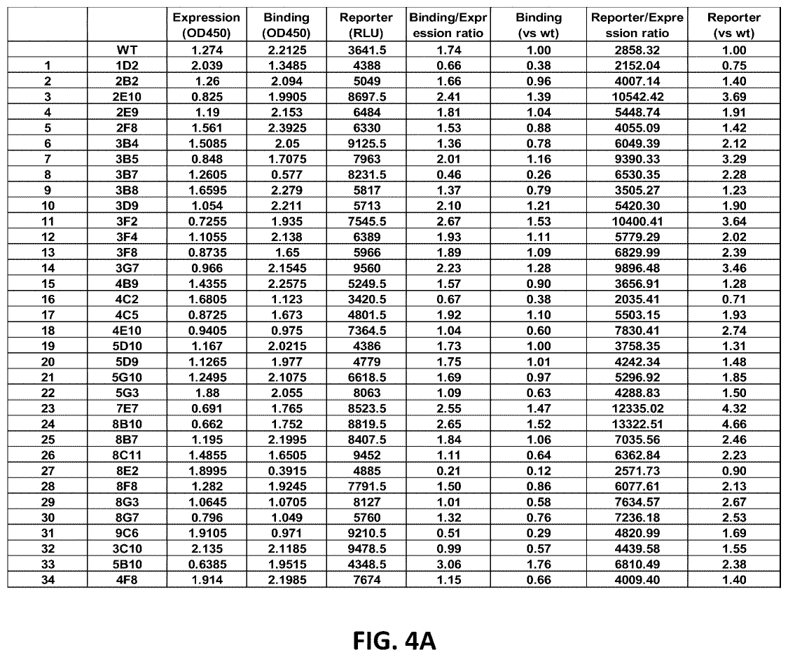

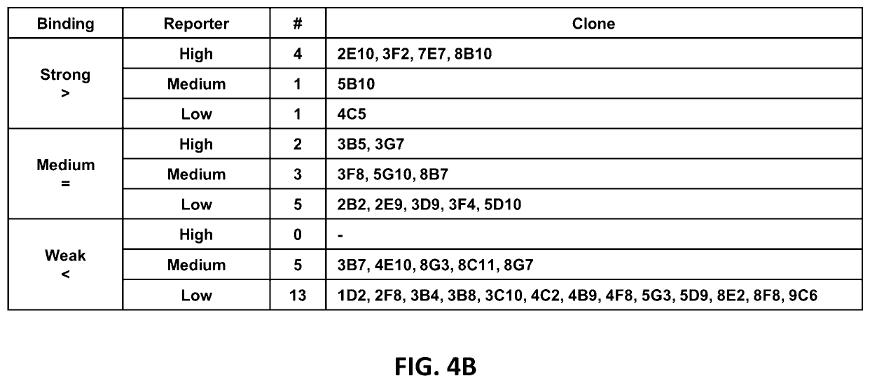

The results are listed in FIG. 4A, and the characteristics of 34 unique 4-1BBL variants are listed in FIG. 4B. 6 clones showed higher 4-1BB binding than wild-type of 4-1BBL, and exhibited different potency on induction of 4-1BB-mediated NF-κB reporter activity. 10 clones showed similar 4-1BB binding activity as compared to wild-type of 4-1BBL, and exhibited different potency on induction of 4-1BB-mediated NF-κB reporter activity. The remaining 18 clones showed relatively weaker 4-1BB-binding activity and lower potency on induction of 4-1BB-mediated NF-κB reporter activity.

As a result, 16 potential candidates with strong or medium 4-1BB-binding activity were selected to construct respective Fc fusion proteins. To increase the diversity of 4-1BBL variants, two candidates with weaker 4-1BB-binding activity (8G3 and 8G7) were also selected to construct respective Fc fusion proteins.

### Example 3. Characterization of 4-1BBL Variants Fused with IgG4 Fc

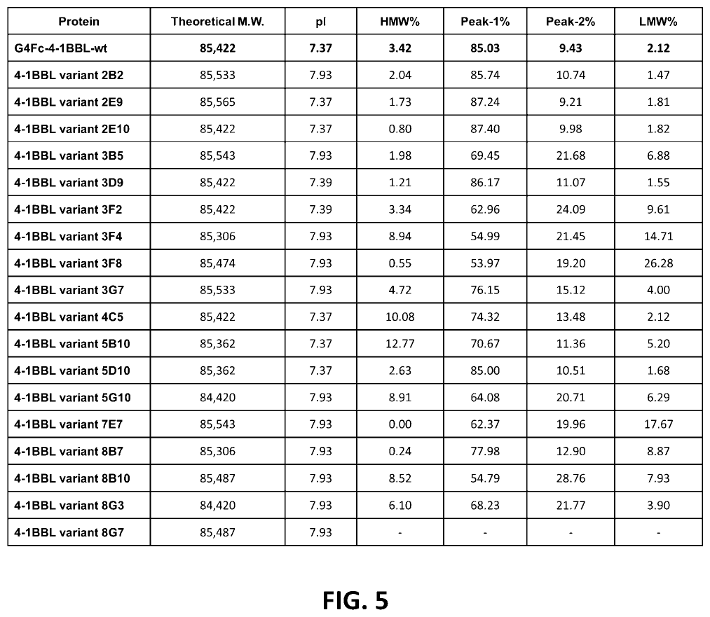

HPLC-SEC (high-performance liquid chromatography-size exclusion chromatography) analysis results of IgG4 Fc (G4Fc) fused 4-1BBL variants are summarized in FIG. 5. Peak-1 represents the trimeric 4-1BBL, and peak-2 represents the dimeric 4-1BBL.

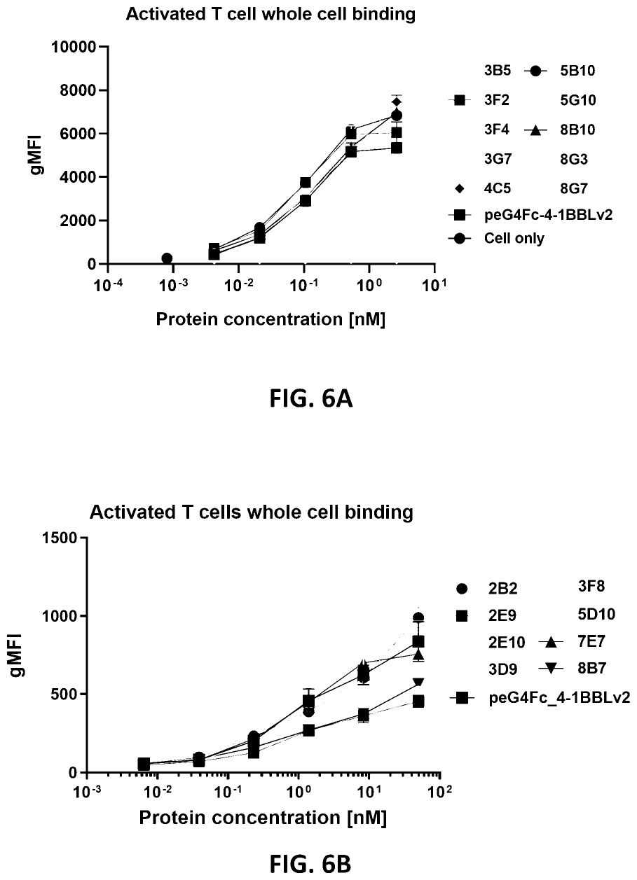

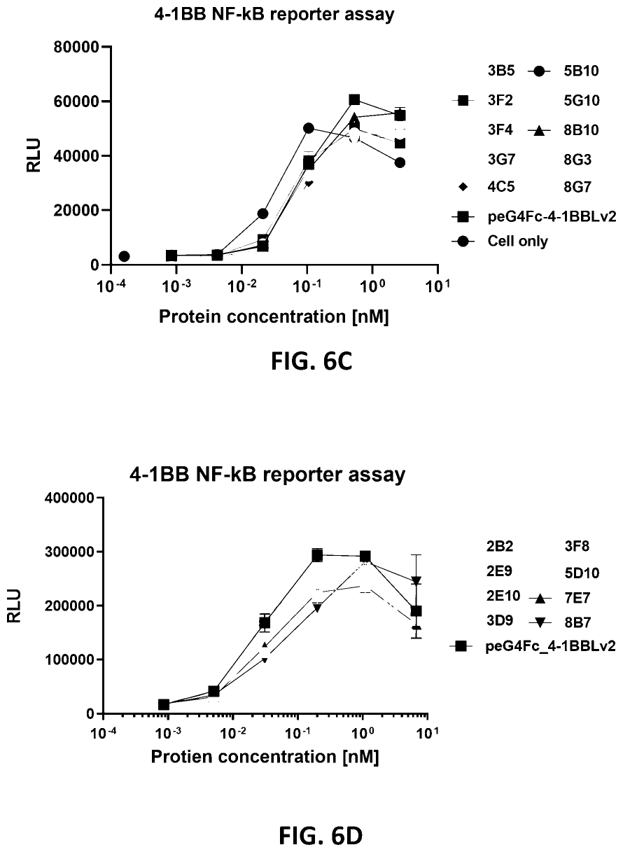

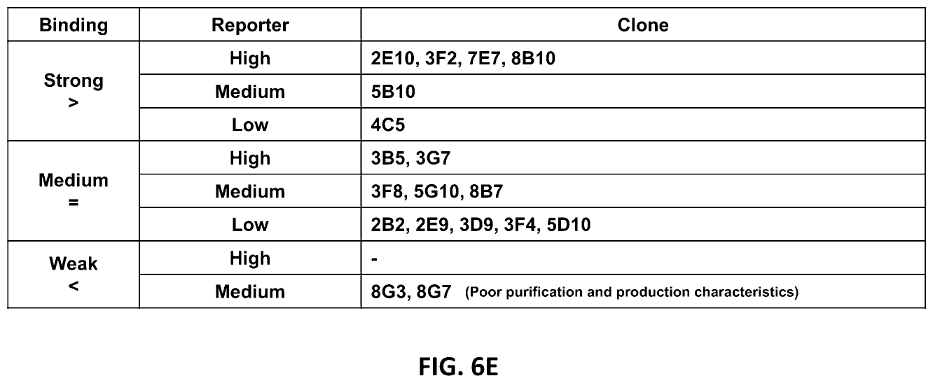

The activated T cell binding activity and 4-1BB-mediated reporter activity of G4Fc-4-1BBL variants were determined by flow cytometry (FIGS. 6A-6B) and reporter assays (FIGS. 6C-6D), respectively.

The activated T cell (whole cell) binding activity was determined as follows. T cells were isolated from a human PBMC donor by Ficoll-Paque® density gradient centrifugation. The isolated T cells were activated by CD3/CD28 Dynabeads® at a cell-to-bead ratio of 1:2 for 4 days to induce 4-1BB expression. 5×10⁴/well of activated T cells were incubated with the G4Fc-4-1BBL variants at indicated concentrations for 30 minutes at 4° C., and then incubated with PE-conjugated anti-human Fc secondary antibody for 30 minutes at 4° C. The binding activity was analyzed by a CytoFlex™ flow cytometer.

With respect to the 4-1BB-mediated NF-κB reporter assay, 5×10⁴/well of transfected NF-κB Jurkat cells expressing 4-1BB were incubated with 5×10⁴/well of FcγRIIb-expressing 293F cells and G4Fc-4-1BBL variants at indicated concentrations for 5 hours at 37° C. The luminance signal was detected to determine the 4-1BB reporter activity.

As shown in FIG. 6E, G4Fc-4-1BBL variants 4C5 and 5B10 showed higher T cell binding activity than wild-type 4-1BBL (peG4Fc_4-1BBLv2 or G4Fc-4-1BBL-wt; SEQ ID NO: 3) and medium to low potency on induction of 4-1BB-mediated NF-κB reporter activity. G4Fc-4-1BBL variants 2B2, 2E9, 3D9, 3F4, and 5D10 showed similar T cell binding activity as compared to wild-type 4-1BBL and low potency on induction of 4-1BB-mediated NF-κB reporter activity. 8G3 and 8G7 were excluded for subsequence experiments due to the poor purification and production titers.

### Example 4. Determination of T Cell Response Potency of Selected G4Fc-4-1BBL Variants-1

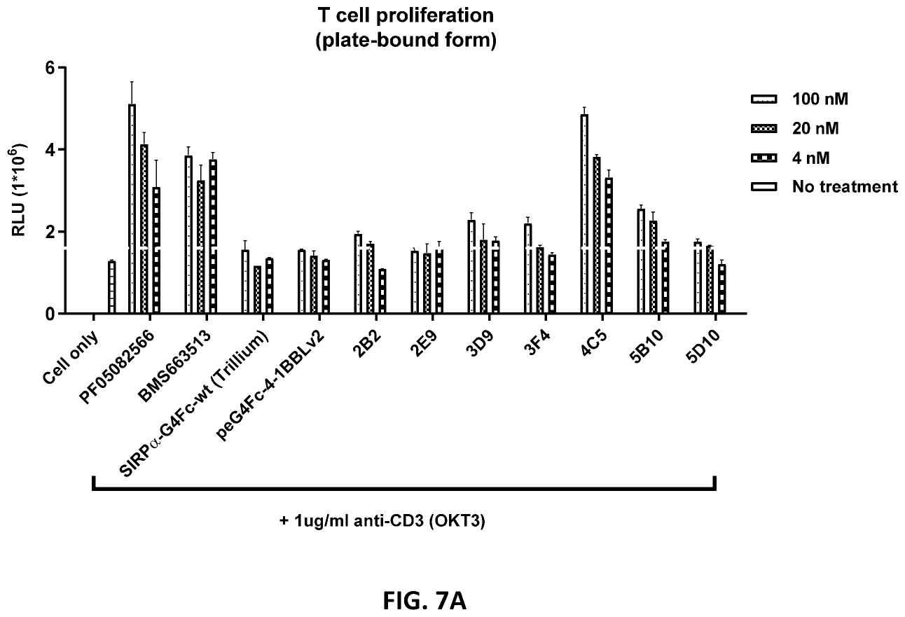

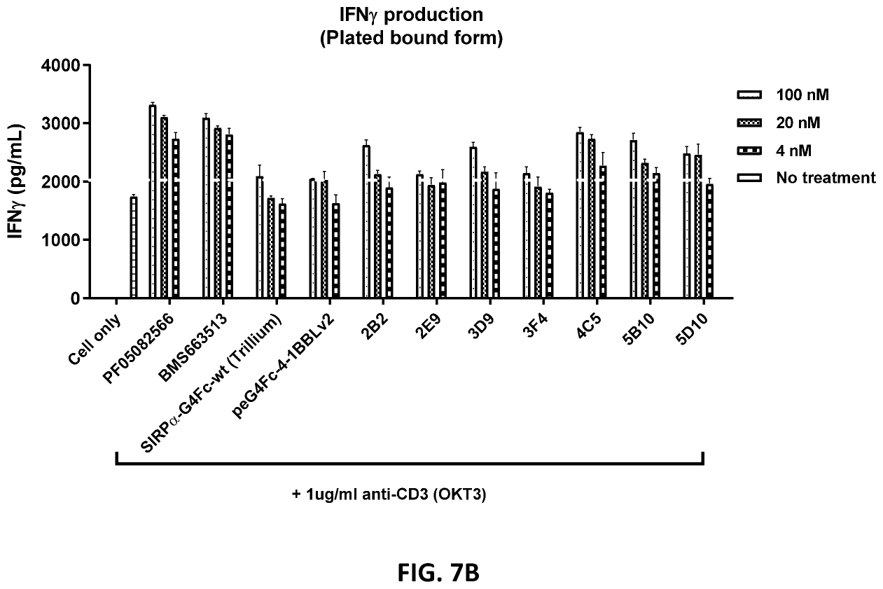

The potency of G4Fc-4-1BBL variants on T cell response was determined by plate-bound format. Briefly, a 96-well plate (U bottom) was coated with G4Fc-4-1BBL variants at indicated concentrations (4-100 nM), and T cell proliferation (FIG. 7A) was determined by the CellTiter-Glo® Cell Viability Assay (Promega). The secretion of IFN-γ (FIG. 7B) was determined by ELISA MAX™ Deluxe Set Human IFN-γ kit (BioLegend) according to manufacturer's protocol.

Specifically, T cells were isolated from a human PBMC donor by Ficoll-Paque® density gradient centrifugation. The isolated T cells were activated by CD3/CD28 Dynabeads® at 1:10 cell-to-beads ratio for 3 days to induce 4-1BB expression. A 96-well plate was pre-coated with 1 μg/ml of anti-CD3 antibody (OKT-3) for 3 hours at 37° C., and then blocked with 1% FBS in PBS for 1 hour at RT. The G4Fc-4-1BBL variants were incubated in the plate at indicated concentrations at 4° C. overnight. After the incubation, 5×10⁴/well of pre-activated T cells were added and the plate was cultured for another 4 days. Supernatant was collected for cytokine detection and T cell proliferation was determined by the CellTiter-Glo® Cell Viability Assay (Promega).

As shown in FIGS. 7A-7B, G4Fc-4-1BBL variants 2B2, 3D9, 4C5, and 5B10 showed higher potency to induce T cell proliferation and cytokine production than wild-type 4-1BBL (peG4Fc_4-1BBLv2; SEQ ID NO: 3) and SIRPα-G4Fc-wt (Trillium; SEQ ID NO: 4).

### Example 5. Determination of T Cell Response Potency of Selected G4Fc-4-1BBL Variants-2

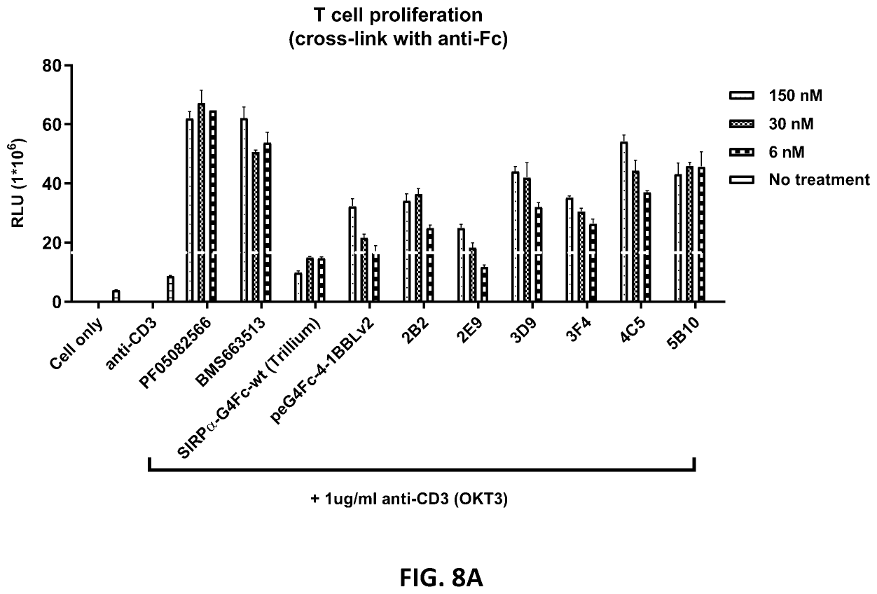

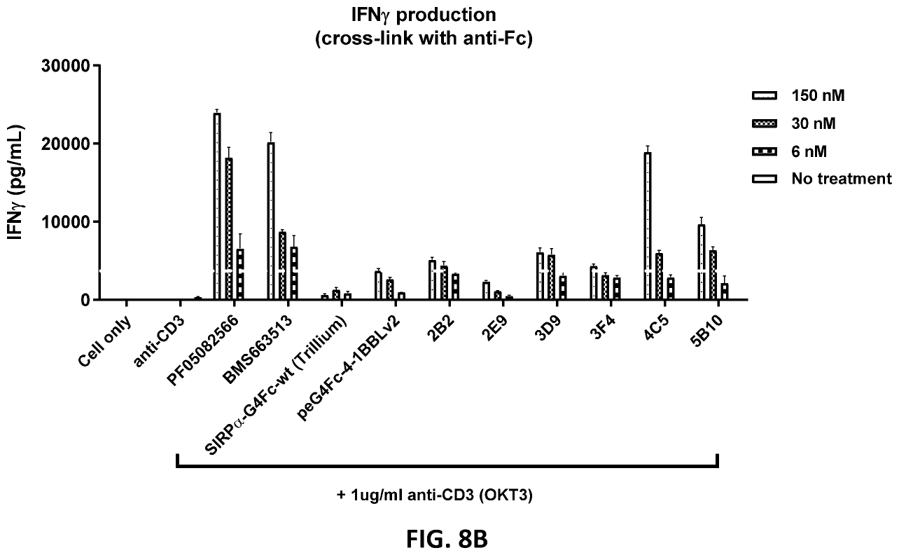

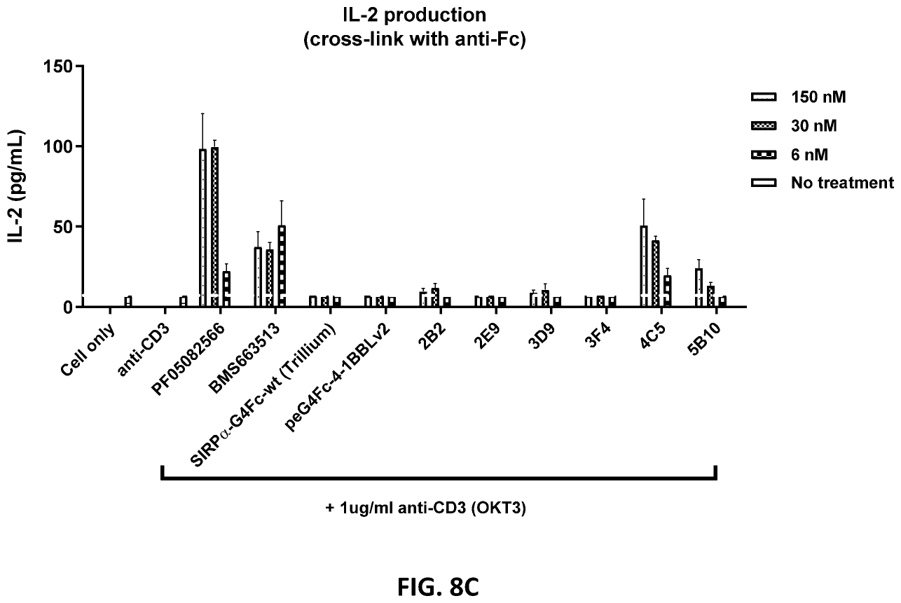

The potency of G4Fc-4-1BBL variants on T cell response was also determined by crosslinking with an anti-human Fc antibody. Briefly, a 96-well plate (U bottom) was coated with an anti-human Fc antibody, and G4Fc-4-1BBL variants were cross-linked through interaction with the pre-coated anti-human Fc at indicated concentrations (6-150 μM of G4Fc-4-1BBL variants). T cell proliferation (FIG. 8A) was determined by the CellTiter-Glo® Cell Viability Assay (Promega). The secretion of cytokines (FIGS. 8B-8C) was determined by ELISA MAXIM Deluxe Set Human IFN-γ and IL-2 kits (BioLegend) according to manufacturer's protocol.

Specifically, T cells were isolated from a human PBMC donor by Ficoll-Paque® density gradient centrifugation. The isolated T cells were activated by CD3/CD28 Dynabeads® at 1:10 cell-to-beads ratio for 3 days to induce 4-1BB expression. A 96-well plate was pre-coated with 1 μg/ml of anti-CD3 antibody (OKT-3) and 3 μg/ml of anti-human Fc for 3 hours at 37° C., and then blocked with 1% FBS in PBS for 1 hour at RT. The G4Fc-4-1BBL variants were incubated in the plate at indicated concentrations at 4° C. overnight. After the incubation, 5×10⁴/well of pre-activated T cells were added and the plate was cultured for another 4 days. Supernatant was collected for cytokine detection and T cell proliferation was determined by the CellTiter-Glo® Cell Viability Assay (Promega).

As shown in FIGS. 8A-8C, G4Fc-4-1BBL variants 2B2, 3D9, 4C5, and 5B10 showed higher potency on induction of T cell proliferation and IFN-γ production than wild-type 4-1BBL (peG4Fc_4-1BBLv2; SEQ ID NO: 3). Further, G4Fc-4-1BBL variants 4C5 and 5B10 showed higher potency on induction of IL-2 production than other tested G4Fc-4-1BBL variants.

### Example 6. Verification of T Cell Binding and Reporter Activities

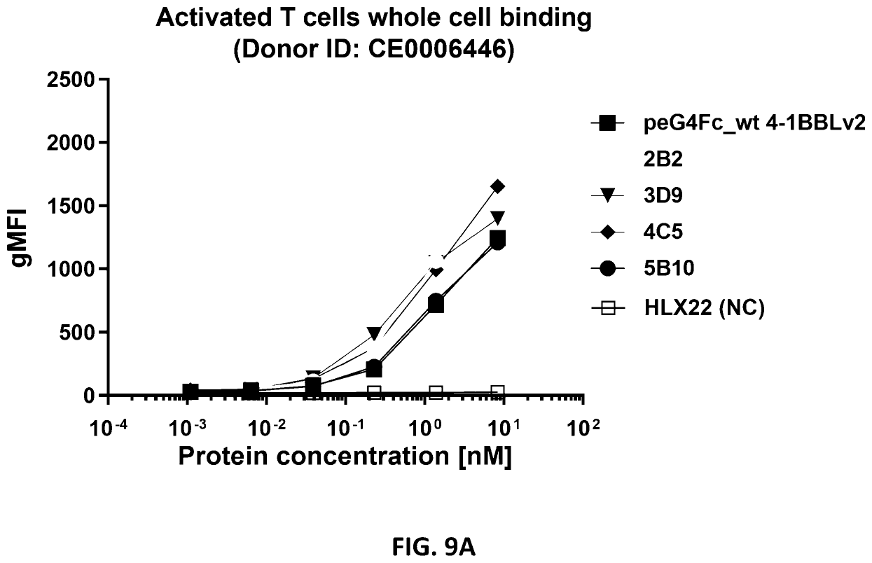

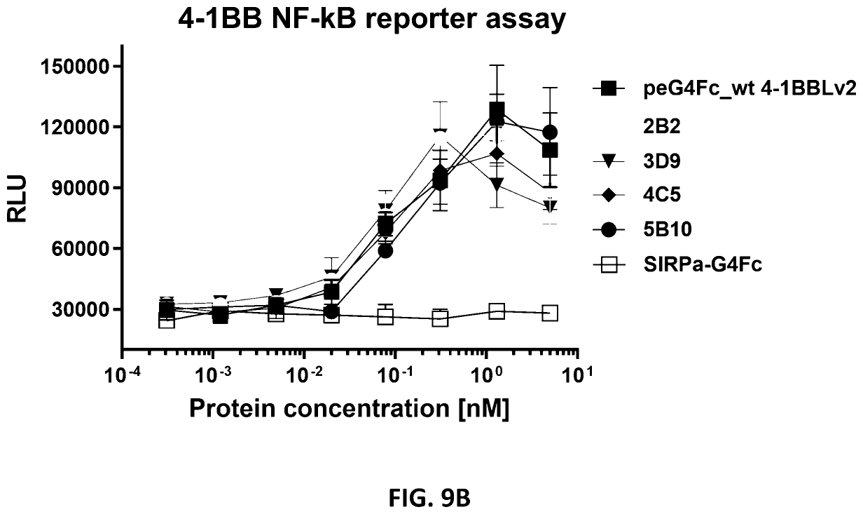

The activated T cell binding activity and 4-1BB-mediated reporter activity of G4Fc-4-1BBL variants 2B2, 3D9, 4C5, and 5B10 were further verified by flow cytometry (FIG. 9A) and reporter assays (FIG. 9B), respectively. The same methods of the activated T cell (whole cell) binding assay and the 4-1BB-mediated NF-κB reporter assay described in Example 3 were performed.

Compared to wild-type of 4-1BBL ((peG4Fc_4-1BBLv2; SEQ ID NO: 3), G4Fc-4-1BBL variants 2B2, 3D9 and 4C5 showed higher T cell binding activity and similar agonist function. Because 4C5 showed poor purification by HPLC-SEC (FIG. 5), G4Fc-4-1BBL variants 2B2 and 3D9 were selected for subsequent experiments.

### Example 7. Determination of T Cell Response Potency of G4Fc-4-1BBL Variants

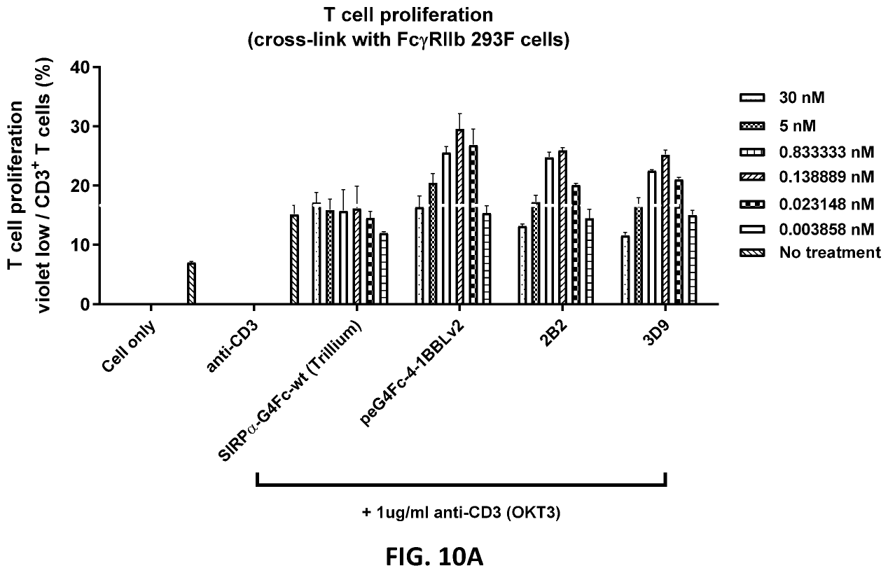

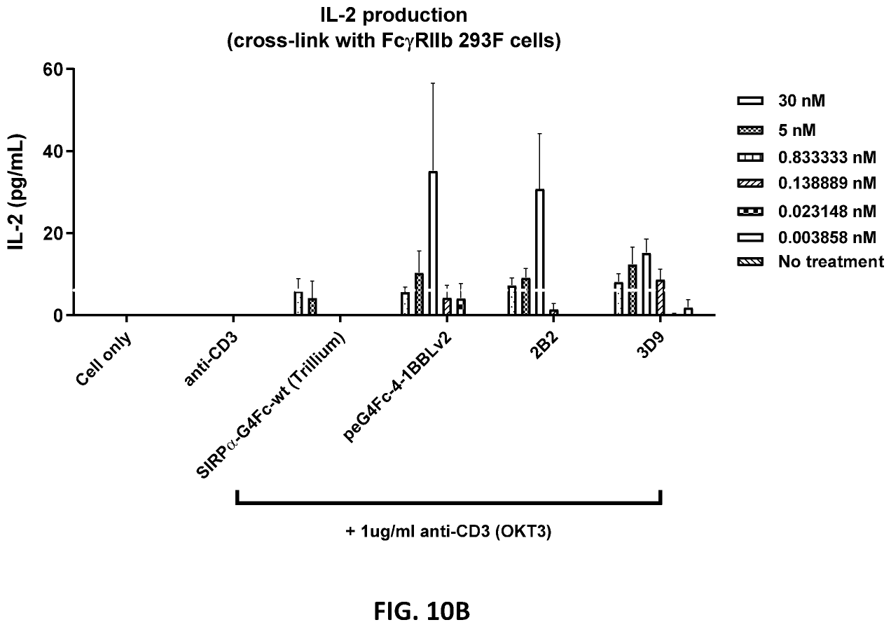

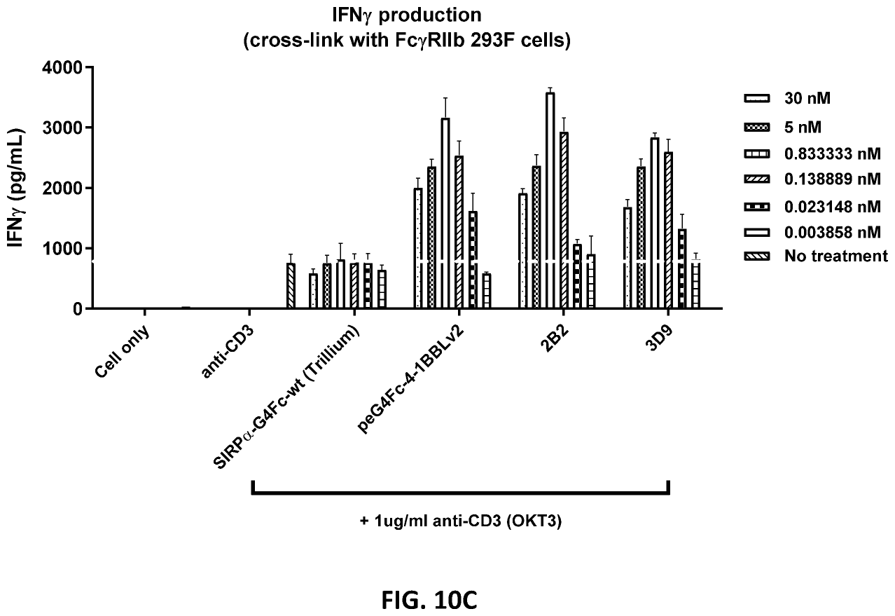

The potency of G4Fc-4-1BBL variants on T cell response was further verified by crosslinking with anti-human Fc antibody. Briefly, G4Fc-4-1BBL variants were cross-linked through interaction with FcγRIIb 293F cells at indicated concentrations (0.003858-30 nM). T cell proliferation (FIG. 10A) was determined by measuring the percentage of Violet^{low} CD3+ T cells by a CytoFlex™ flow cytometer. The secretion of cytokines (FIGS. 10B-10C) was determined by ELISA MAX™ Deluxe Set Human IFN-γ and IL-2 kits (BioLegend) according to manufacturer's protocol.

Specifically, T cells were isolated from a human PBMC donor by Ficoll-Paque® density gradient centrifugation. The isolated T cells were activated by CD3/CD28 Dynabeads® at 1:1 cell-to-beads ratio for 1 day to induce 4-1BB expression. A 96-well plate (U bottom) was pre-coated with 1 μg/ml of anti-CD3 antibody (OKT-3) for 3 hours at 37° C., and then blocked with 1% FBS in PBS for 1 hour at RT. The G4Fc-4-1BBL variants at indicated concentrations were incubated with Mitomycin C-treated FcγRIIb 293F and CellTrace™ Violet-labeled CD3+ T cells for 3 Days. Supernatant was collected for cytokine detection and T cell proliferation was determined by a CytoFlex™ flow cytometer.

Compared to wild-type G4Fc-4-1BBL (peG4Fc_4-1BBLv2; SEQ ID NO: 3), 2B2 and 3D9 showed higher T cell binding activity and decreased ability to induce T cell proliferation and cytokine release.

### Example 8. Determination of Hepatotoxicity Risk of G4Fc-4-1BBL Variants

The potential hepatotoxicity risk was determined by induction of IL-27 and TNF-α production. Specifically, 2.5× 104/well of MDM (monocyte-derived macrophage) cells were stimulated by 1.5 μg/ml of LPS and incubated with G4Fc-4-1BBL variants at indicated concentrations for 48 hours. Production of IL-27 and TNF-α was determined by Human IL-27 DuoSet ELISA kit (R&D) and ELISA MAX™ Deluxe Set Human TNF-α kit (BioLegend) according to manufacturer's protocol.

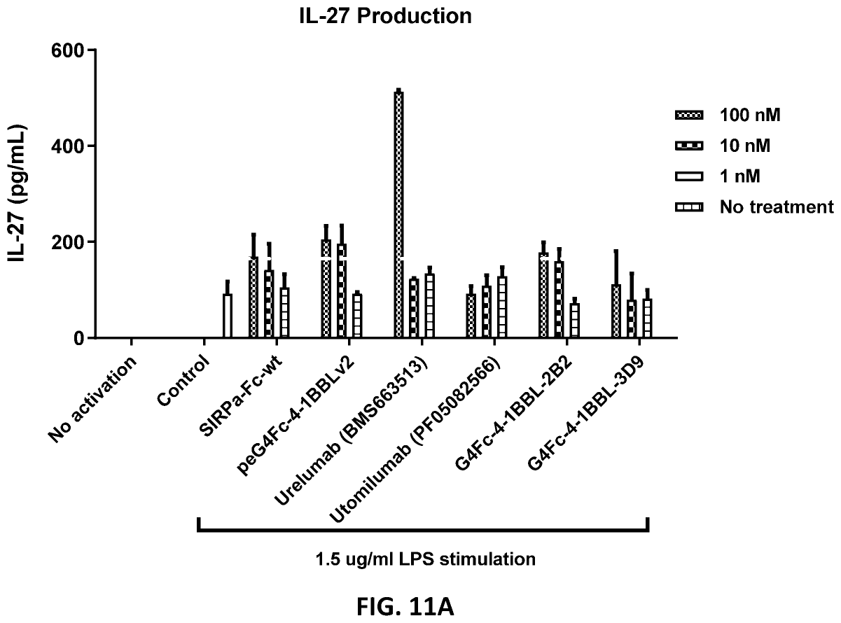

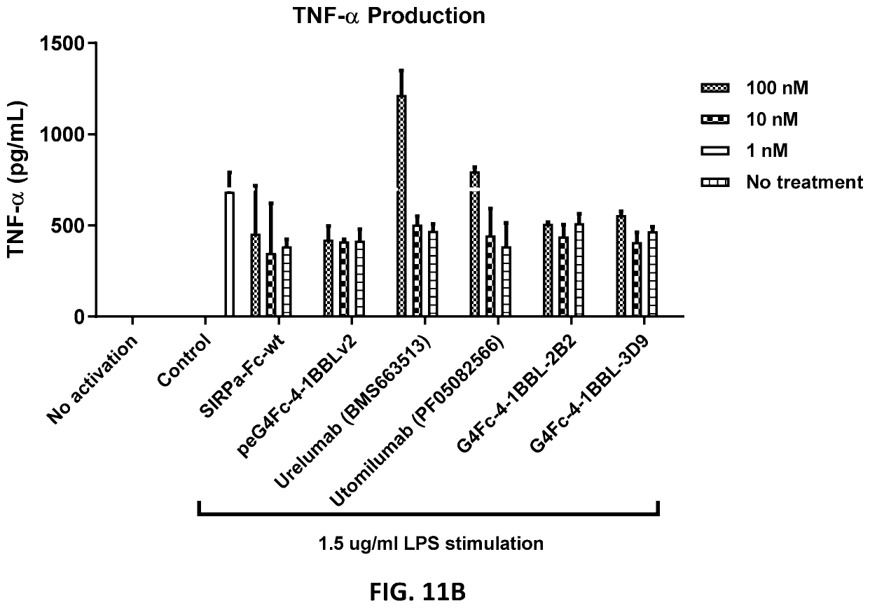

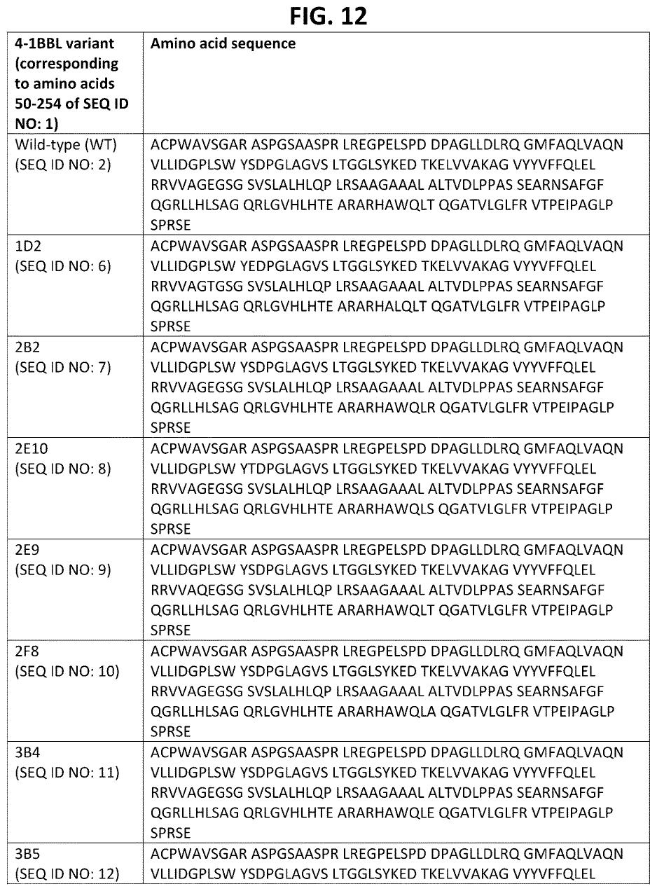

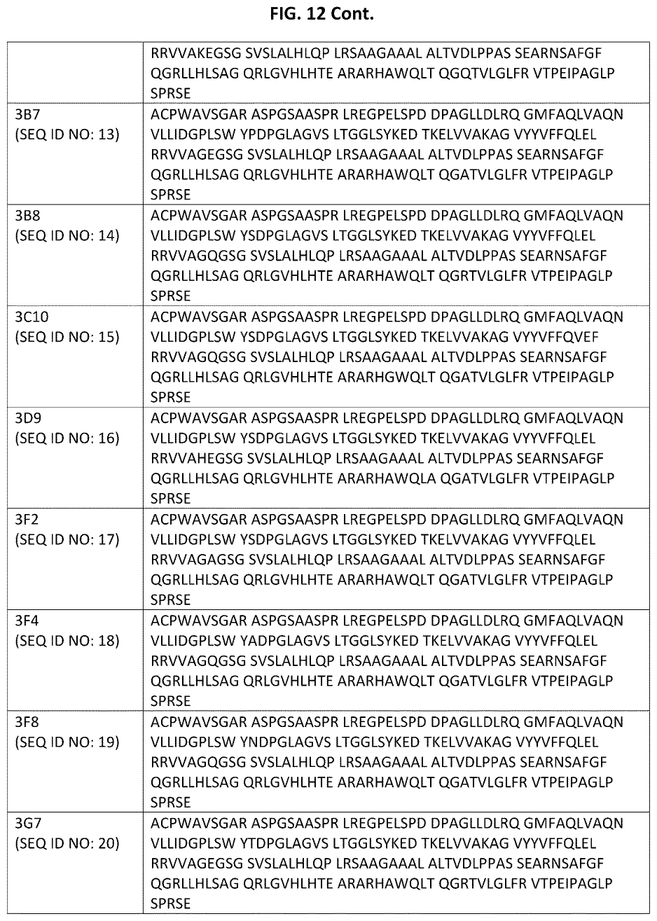

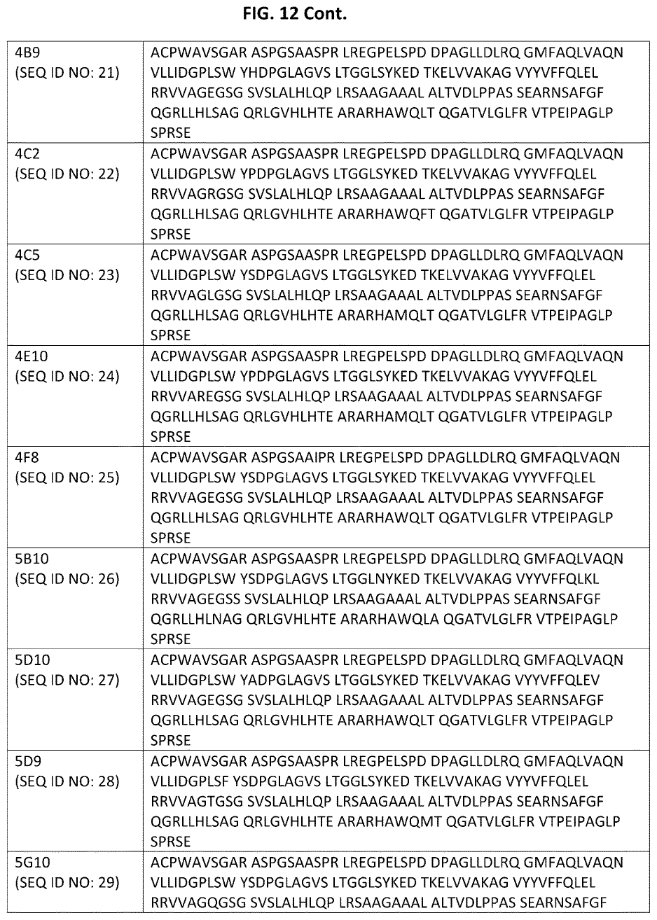

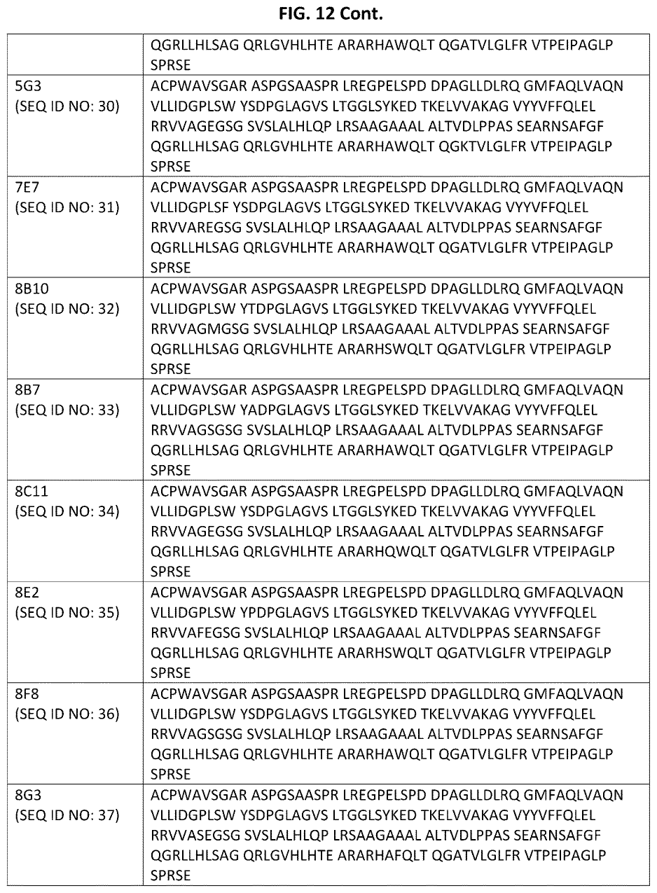

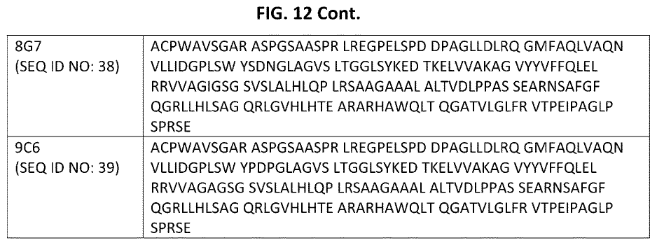

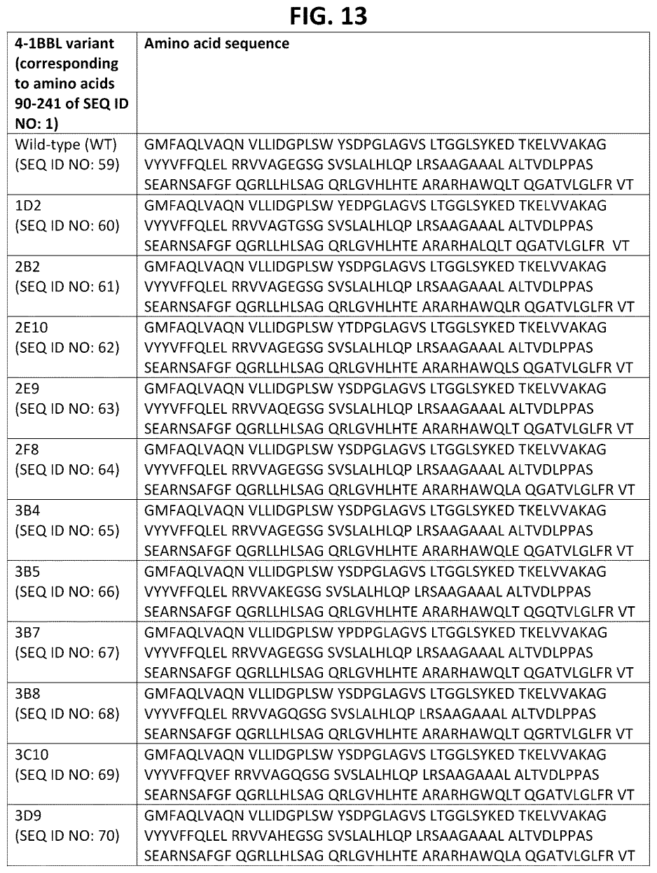

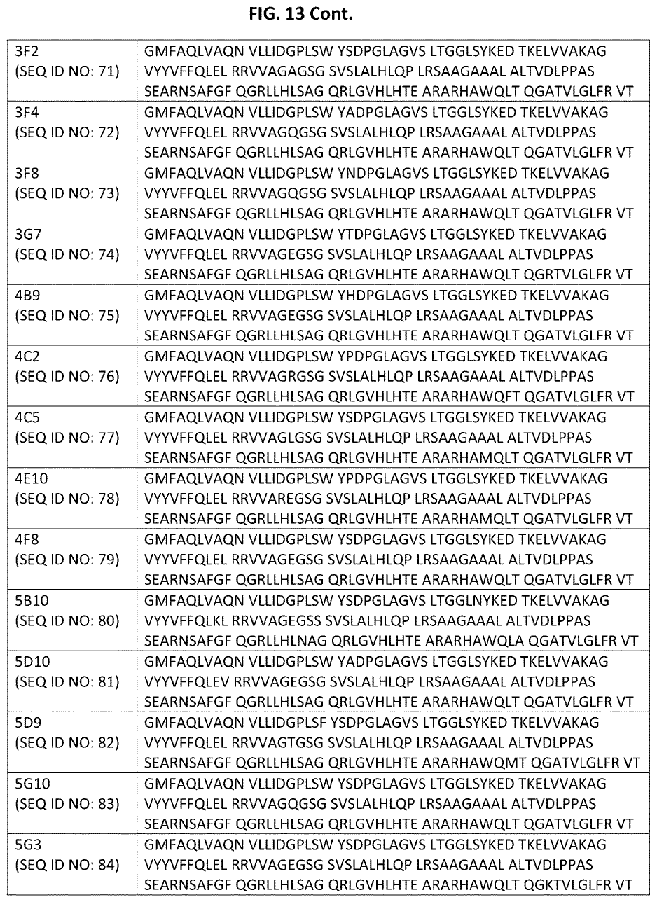

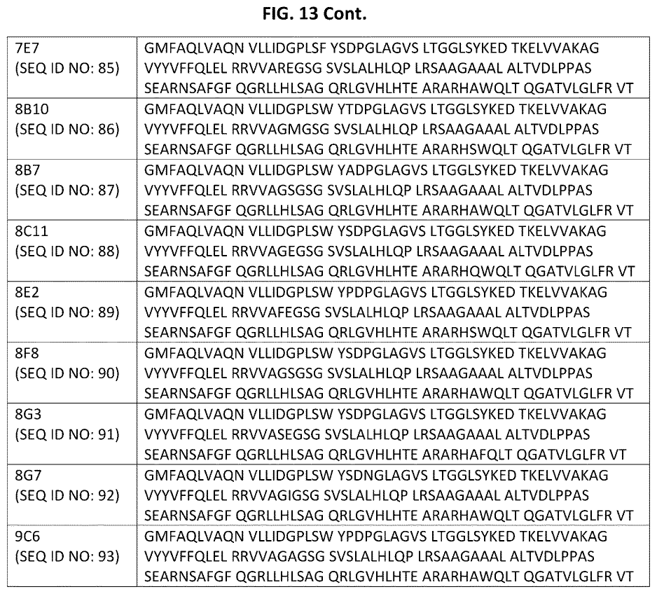

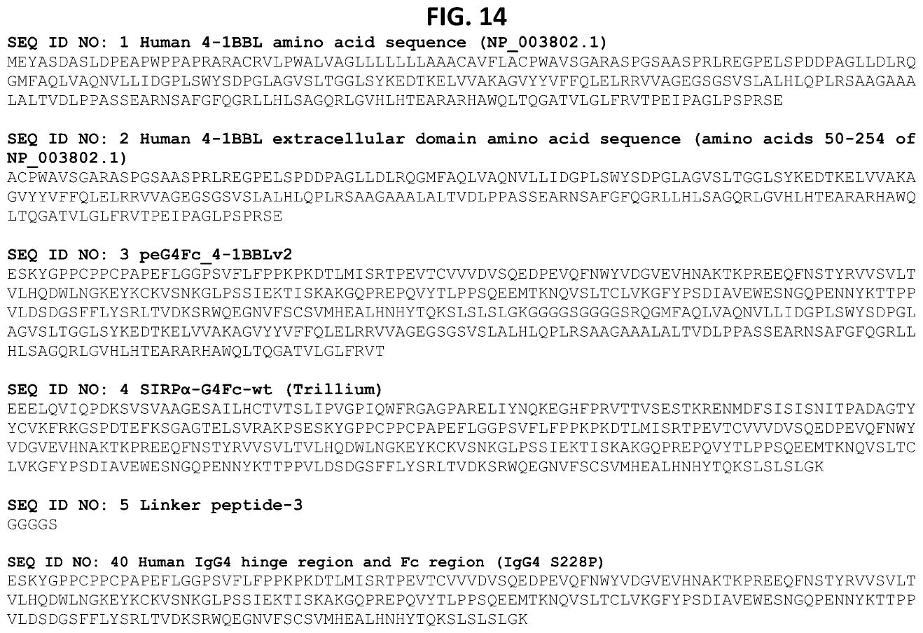

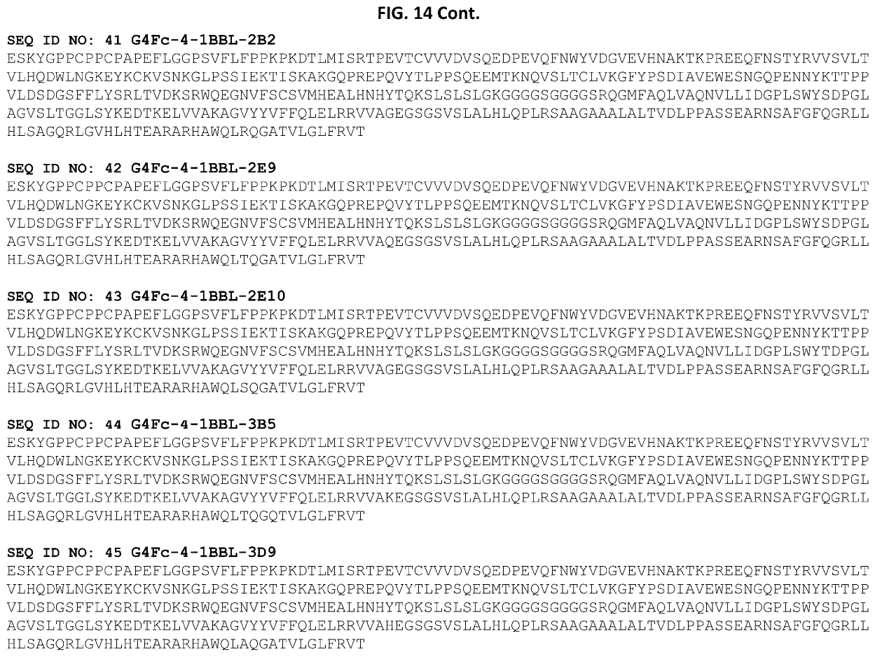

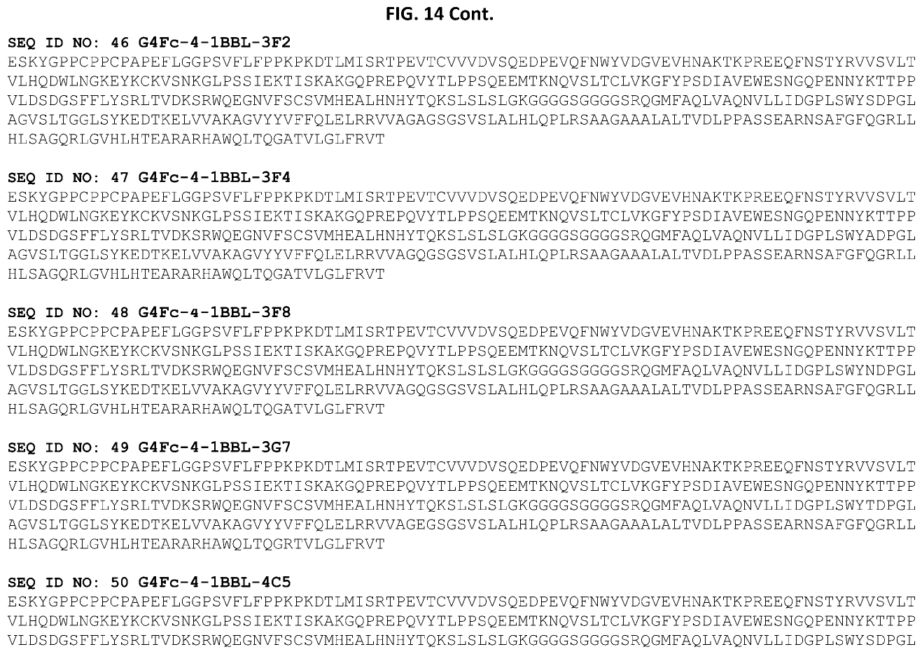

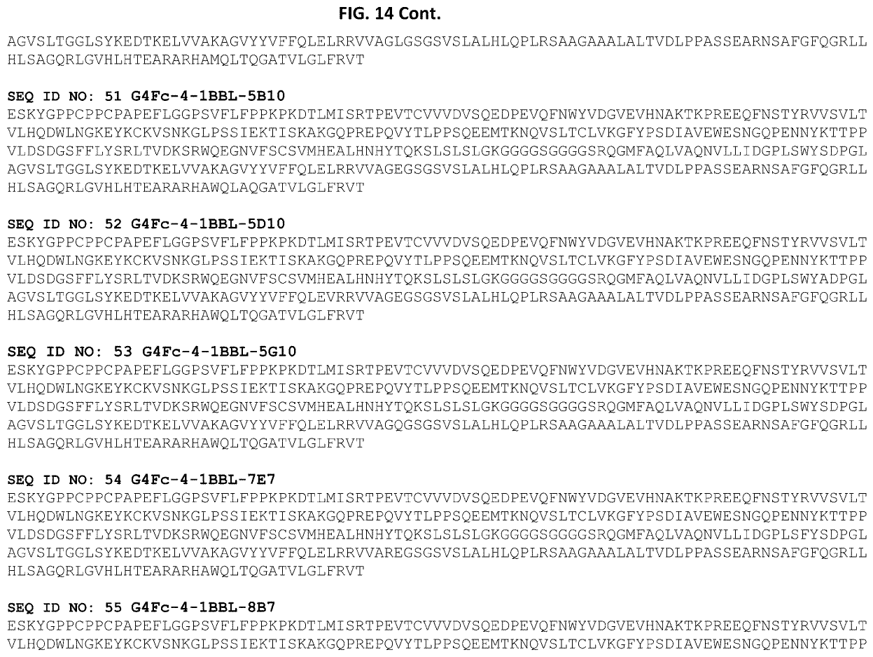

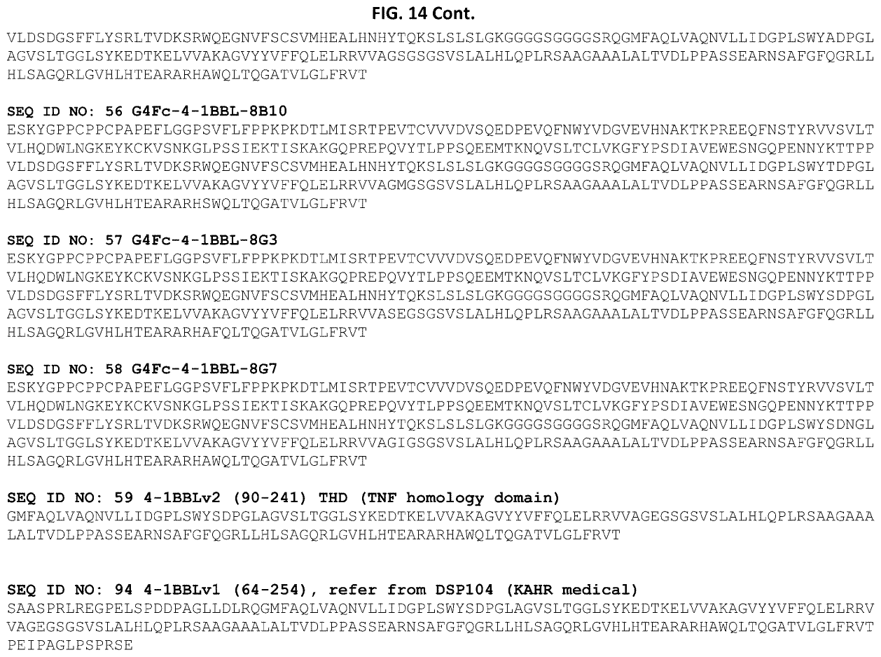

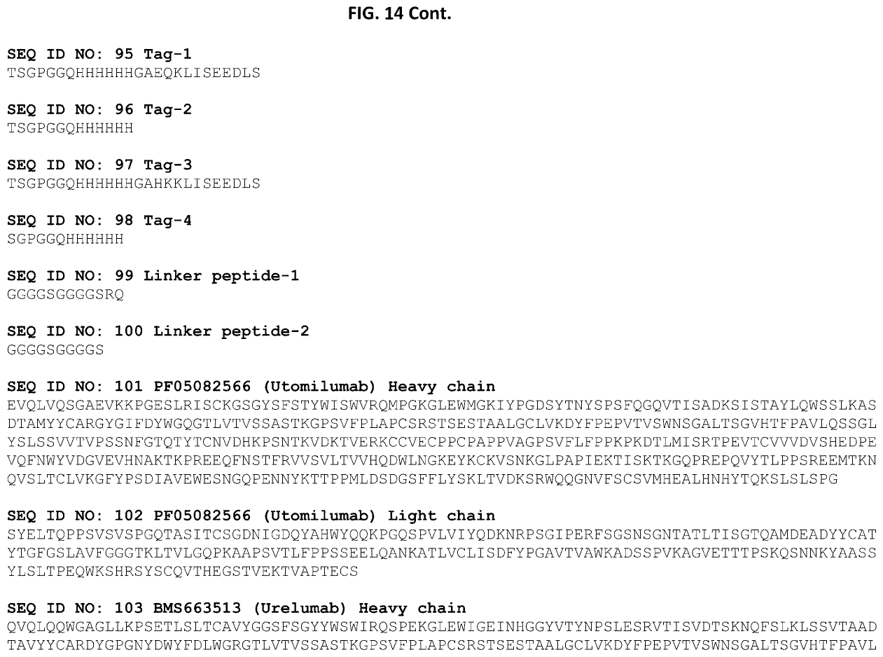

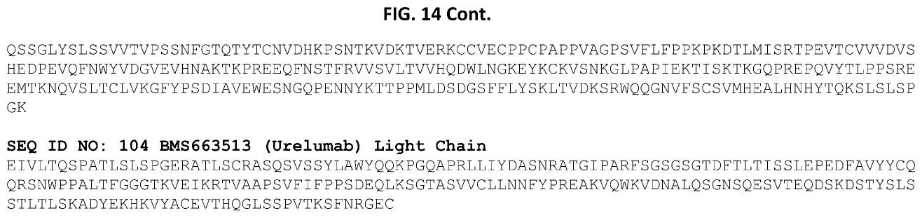

As shown in FIGS. 11A-11B, G4Fc-4-1BBL variants 2B2 and 3D9 did not enhance the IL-27 and TNF-α production relative to wild-type G4Fc-4-1BBL (peG4Fc_4-1BBLv2; SEQ ID NO: 3). The results indicate that 4-1BBL variants 2B2 and 3D9 do not show a risk on liver toxicity.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An engineered 4-1BBL polypeptide comprising an amino acid sequence that is at least 80% identical to SEQ ID NO: 2 or SEQ ID NO: 59, wherein the engineered 4-1BBL polypeptide comprises one or more amino acid mutations at AA′ loop, CD loop, and/or GH loop.

2. The engineered 4-1BBL polypeptide of claim 1, wherein the amino acid that corresponds to S62 of SEQ ID NO: 2 is E, T, P, A, N, T, or H.

3. The engineered 4-1BBL polypeptide of claim 1 or 2, comprising one or more of the following:
(a) the amino acid that corresponds to G106 of SEQ ID NO: 2 is Q, K, H, R, F, or S; and
(b) the amino acid that corresponds to E107 of SEQ ID NO: 2 is T, Q, A, R, L, M, S, or I.

4. The engineered 4-1BBL polypeptide of any one of claims 1-3, comprising one or more of the following:
(a) the amino acid that corresponds to A176 of SEQ ID NO: 2 is S or Q;
(b) the amino acid that corresponds to W177 of SEQ ID NO: 2 is L, M, or F;
(c) the amino acid that corresponds to L179 of SEQ ID NO: 2 is F, A, or M;
(d) the amino acid that corresponds to T180 of SEQ ID NO: 2 is R, S, A, or E; and
(e) the amino acid that corresponds to A183 of SEQ ID NO: 2 is Q, R, or K.

5. The engineered 4-1BBL polypeptide of any one of claims 1-4, comprising one or more of the following:
(a) the amino acid that corresponds to W60 of SEQ ID NO: 2 is F; and
(b) the amino acid that corresponds to P64 of SEQ ID NO: 2 is N.

6. The engineered 4-1BBL polypeptide of any one of claims 1-5, wherein the amino acid that corresponds to L100 of SEQ ID NO: 2 is V.

7. The engineered 4-1BBL polypeptide of any one of claims 1-6, further comprising one or more of the following:
(a) the amino acid that corresponds to S18 of SEQ ID NO: 2 is I; and
(b) the amino acid that corresponds to L98 of SEQ ID NO: 2 is V.

8. The engineered 4-1BBL polypeptide of any one of claims 1-7, comprising one or more of the following:
(a) the amino acid that corresponds to position 61 of SEQ ID NO: 2 is Y;
(b) the amino acid that corresponds to position 63 of SEQ ID NO: 2 is D;
(c) the amino acid that corresponds to position 65 of SEQ ID NO: 2 is G;
(d) the amino acid that corresponds to position 66 of SEQ ID NO: 2 is L;
(e) the amino acid that corresponds to position 101 of SEQ ID NO: 2 is R;
(f) the amino acid that corresponds to position 102 of SEQ ID NO: 2 is R;
(g) the amino acid that corresponds to position 103 of SEQ ID NO: 2 is V;
(h) the amino acid that corresponds to position 104 of SEQ ID NO: 2 is V;
(i) the amino acid that corresponds to position 105 of SEQ ID NO: 2 is A;
(j) the amino acid that corresponds to position 178 of SEQ ID NO: 2 is Q; and
(k) the amino acid that corresponds to position 181 of SEQ ID NO: 2 is Q.

9. The engineered 4-1BBL polypeptide of any one of claims 1-8, comprising an amino acid sequence that is at least 85%, 90%, 95%, or 100% identical to SEQ ID NO: 2, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39.

10. The engineered 4-1BBL polypeptide of any one of claims 1-8, comprising an amino acid sequence that is at least 85%, 90%, 95%, or 100% identical to SEQ ID NO: 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, or 93.

11. An engineered 4-1BBL polypeptide comprising an amino acid sequence that is at least 80% identical to SEQ ID NO: 2, 6, 59, or 60, wherein the polypeptide comprises one or more of the following:
(a) the amino acid that corresponds to S62 of SEQ ID NO: 2 is E;
(b) the amino acid that corresponds to E107 of SEQ ID NO: 2 is T; and
(c) the amino acid that corresponds to W177 of SEQ ID NO: 2 is L.

12. The engineered 4-1BBL polypeptide of claim 11, comprising an amino acid sequence that is at least 90% identical to SEQ ID NO: 6 or 60.

13. An engineered 4-1BBL polypeptide comprising an amino acid sequence that is at least 80% identical to SEQ ID NO: 2, 7, 59, or 61, wherein the amino acid that corresponds to T180 of SEQ ID NO: 2 is R.

14. The engineered 4-1BBL polypeptide of claim 13, comprising an amino acid sequence that is at least 90% identical to SEQ ID NO: 7 or 61.

15. An engineered 4-1BBL polypeptide comprising an amino acid sequence that is at least 80% identical to SEQ ID NO: 2, 8, 59, or 62, wherein the polypeptide comprises one or more of the following:
(a) the amino acid that corresponds to S62 of SEQ ID NO: 2 is T; and
(b) the amino acid that corresponds to T180 of SEQ ID NO: 2 is S.

16. The engineered 4-1BBL polypeptide of claim 15, comprising an amino acid sequence that is at least 90% identical to SEQ ID NO: 8 or 62.

17. An engineered 4-1BBL polypeptide comprising an amino acid sequence that is at least 80% identical to SEQ ID NO: 2, 9, 59, or 63, wherein the amino acid that corresponds to G106 of SEQ ID NO: 2 is Q.

18. The engineered 4-1BBL polypeptide of claim 17, comprising an amino acid sequence that is at least 90% identical to SEQ ID NO: 9 or 63.

19. An engineered 4-1BBL polypeptide comprising an amino acid sequence that is at least 80% identical to SEQ ID NO: 2, 10, 59, or 64, wherein the amino acid that corresponds to T180 of SEQ ID NO: 2 is A.

20. The engineered 4-1BBL polypeptide of claim 19, comprising an amino acid sequence that is at least 90% identical to SEQ ID NO: 10 or 64.

21. An engineered 4-1BBL polypeptide comprising an amino acid sequence that is at least 80% identical to SEQ ID NO: 2, 11, 59, or 65, wherein the amino acid that corresponds to T180 of SEQ ID NO: 2 is E.

22. The engineered 4-1BBL polypeptide of claim 21, comprising an amino acid sequence that is at least 90% identical to SEQ ID NO: 11 or 65.

23. An engineered 4-1BBL polypeptide comprising an amino acid sequence that is at least 80% identical to SEQ ID NO: 2, 12, 59, or 66, wherein the polypeptide comprises one or more of the following:
(a) the amino acid that corresponds to G106 of SEQ ID NO: 2 is K; and
(b) the amino acid that corresponds to A183 of SEQ ID NO: 2 is Q.

24. The engineered 4-1BBL polypeptide of claim 23, comprising an amino acid sequence that is at least 90% identical to SEQ ID NO: 12 or 66.

25. An engineered 4-1BBL polypeptide comprising an amino acid sequence that is at least 80% identical to SEQ ID NO: 2, 13, 59, or 67, wherein the amino acid that corresponds to S62 of SEQ ID NO: 2 is P.

26. The engineered 4-1BBL polypeptide of claim 25, comprising an amino acid sequence that is at least 90% identical to SEQ ID NO: 13 or 67.

27. An engineered 4-1BBL polypeptide comprising an amino acid sequence that is at least 80% identical to SEQ ID NO: 2, 14, 59, or 68, wherein the polypeptide comprises one or more of the following:
(a) the amino acid that corresponds to E107 of SEQ ID NO: 2 is Q; and
(b) the amino acid that corresponds to A183 of SEQ ID NO: 2 is R.

28. The engineered 4-1BBL polypeptide of claim 27, comprising an amino acid sequence that is at least 90% identical to SEQ ID NO: 14 or 68.

29. An engineered 4-1BBL polypeptide comprising an amino acid sequence that is at least 80% identical to SEQ ID NO: 2, 15, 59, or 69, wherein the amino acid that corresponds to L98 of SEQ ID NO: 2 is V.

30. The engineered 4-1BBL polypeptide of claim 29, comprising an amino acid sequence that is at least 90% identical to SEQ ID NO: 15 or 69.

31. An engineered 4-1BBL polypeptide comprising an amino acid sequence that is at least 80% identical to SEQ ID NO: 2, 16, 59, or 70, wherein the polypeptide comprises one or more of the following:
(a) the amino acid that corresponds to G106 of SEQ ID NO: 2 is H; and
(b) the amino acid that corresponds to T180 of SEQ ID NO: 2 is A.

32. The engineered 4-1BBL polypeptide of claim 31, comprising an amino acid sequence that is at least 90% identical to SEQ ID NO: 16 or 70.

33. An engineered 4-1BBL polypeptide comprising an amino acid sequence that is at least 80% identical to SEQ ID NO: 2, 17, 59, or 71, wherein the amino acid that corresponds to E107 of SEQ ID NO: 2 is A.

34. The engineered 4-1BBL polypeptide of claim 33, comprising an amino acid sequence that is at least 90% identical to SEQ ID NO: 17 or 71.

35. An engineered 4-1BBL polypeptide comprising an amino acid sequence that is at least 80% identical to SEQ ID NO: 2, 18, 59, or 72, wherein the polypeptide comprises one or more of the following:
(a) the amino acid that corresponds to S62 of SEQ ID NO: 2 is A; and
(b) the amino acid that corresponds to E107 of SEQ ID NO: 2 is Q.

36. The engineered 4-1BBL polypeptide of claim 35, comprising an amino acid sequence that is at least 90% identical to SEQ ID NO: 18 or 72.

37. An engineered 4-1BBL polypeptide comprising an amino acid sequence that is at least 80% identical to SEQ ID NO: 2, 19, 59, or 73, wherein the polypeptide comprises one or more of the following:
(a) the amino acid that corresponds to S62 of SEQ ID NO: 2 is N; and
(b) the amino acid that corresponds to E107 of SEQ ID NO: 2 is Q.

38. The engineered 4-1BBL polypeptide of claim 37, comprising an amino acid sequence that is at least 90% identical to SEQ ID NO: 19 or 73.

39. An engineered 4-1BBL polypeptide comprising an amino acid sequence that is at least 80% identical to SEQ ID NO: 2, 20, 59, or 74, wherein the polypeptide comprises one or more of the following:
(a) the amino acid that corresponds to S62 of SEQ ID NO: 2 is T; and
(b) the amino acid that corresponds to A183 of SEQ ID NO: 2 is R.

40. The engineered 4-1BBL polypeptide of claim 39, comprising an amino acid sequence that is at least 90% identical to SEQ ID NO: 20 or 74.

41. An engineered 4-1BBL polypeptide comprising an amino acid sequence that is at least 80% identical to SEQ ID NO: 2, 21, 59, or 75, wherein the amino acid that corresponds to S62 of SEQ ID NO: 2 is H.

42. The engineered 4-1BBL polypeptide of claim 41, comprising an amino acid sequence that is at least 90% identical to SEQ ID NO: 21 or 75.

43. An engineered 4-1BBL polypeptide comprising an amino acid sequence that is at least 80% identical to SEQ ID NO: 2, 22, 59, or 76, wherein the polypeptide comprises one or more of the following:
(a) the amino acid that corresponds to S62 of SEQ ID NO: 2 is P;
(b) the amino acid that corresponds to E107 of SEQ ID NO: 2 is R; and
(c) the amino acid that corresponds to L179 of SEQ ID NO: 2 is F.

44. The engineered 4-1BBL polypeptide of claim 43, comprising an amino acid sequence that is at least 90% identical to SEQ ID NO: 22 or 76.

45. An engineered 4-1BBL polypeptide comprising an amino acid sequence that is at least 80% identical to SEQ ID NO: 2, 23, 59, or 77, wherein the polypeptide comprises one or more of the following:
(a) the amino acid that corresponds to E107 of SEQ ID NO: 2 is L; and
(b) the amino acid that corresponds to W177 of SEQ ID NO: 2 is M.

46. The engineered 4-1BBL polypeptide of claim 45, comprising an amino acid sequence that is at least 90% identical to SEQ ID NO: 23 or 77.

47. An engineered 4-1BBL polypeptide comprising an amino acid sequence that is at least 80% identical to SEQ ID NO: 2, 24, 59, or 78, wherein the polypeptide comprises one or more of the following:
(a) the amino acid that corresponds to S62 of SEQ ID NO: 2 is P;
(b) the amino acid that corresponds to G106 of SEQ ID NO: 2 is R; and
(c) the amino acid that corresponds to W177 of SEQ ID NO: 2 is M.

48. The engineered 4-1BBL polypeptide of claim 47, comprising an amino acid sequence that is at least 90% identical to SEQ ID NO: 24 or 78.

49. An engineered 4-1BBL polypeptide comprising an amino acid sequence that is at least 80% identical to SEQ ID NO: 2, 25, 59, or 79, wherein the amino acid that corresponds to S18 of SEQ ID NO: 2 is I.

50. The engineered 4-1BBL polypeptide of claim 49, comprising an amino acid sequence that is at least 90% identical to SEQ ID NO: 25 or 79.

51. An engineered 4-1BBL polypeptide comprising an amino acid sequence that is at least 80% identical to SEQ ID NO: 2, 26, 59, or 80, wherein the amino acid that corresponds to L179 of SEQ ID NO: 2 is A.

52. The engineered 4-1BBL polypeptide of claim 51, comprising an amino acid sequence that is at least 90% identical to SEQ ID NO: 26 or 80.

53. An engineered 4-1BBL polypeptide comprising an amino acid sequence that is at least 80% identical to SEQ ID NO: 2, 27, 59, or 81, wherein the polypeptide comprises one or more of the following:
(a) the amino acid that corresponds to S62 of SEQ ID NO: 2 is A; and
(b) the amino acid that corresponds to L100 of SEQ ID NO: 2 is V.

54. The engineered 4-1BBL polypeptide of claim 53, comprising an amino acid sequence that is at least 90% identical to SEQ ID NO: 27 or 81.

55. An engineered 4-1BBL polypeptide comprising an amino acid sequence that is at least 80% identical to SEQ ID NO: 2, 28, 59, or 82, wherein the polypeptide comprises one or more of the following:
(a) the amino acid that corresponds to W60 of SEQ ID NO: 2 is F;
(b) the amino acid that corresponds to E107 of SEQ ID NO: 2 is T; and
(c) the amino acid that corresponds to L179 of SEQ ID NO: 2 is M.

56. The engineered 4-1BBL polypeptide of claim 55, comprising an amino acid sequence that is at least 90% identical to SEQ ID NO: 28 or 82.

57. An engineered 4-1BBL polypeptide comprising an amino acid sequence that is at least 80% identical to SEQ ID NO: 2, 29, 59, or 83, wherein the amino acid that corresponds to E107 of SEQ ID NO: 2 is Q.

58. The engineered 4-1BBL polypeptide of claim 57, comprising an amino acid sequence that is at least 90% identical to SEQ ID NO: 29 or 83.

59. An engineered 4-1BBL polypeptide comprising an amino acid sequence that is at least 80% identical to SEQ ID NO: 2, 30, 59, or 84, wherein the amino acid that corresponds to A183 of SEQ ID NO: 2 is K.

60. The engineered 4-1BBL polypeptide of claim 59, comprising an amino acid sequence that is at least 90% identical to SEQ ID NO: 30 or 84.

61. An engineered 4-1BBL polypeptide comprising an amino acid sequence that is at least 80% identical to SEQ ID NO: 2, 31, 59, or 85, wherein the polypeptide comprises one or more of the following:
(a) the amino acid that corresponds to W60 of SEQ ID NO: 2 is F; and
(b) the amino acid that corresponds to G106 of SEQ ID NO: 2 is R.

62. The engineered 4-1BBL polypeptide of claim 61, comprising an amino acid sequence that is at least 90% identical to SEQ ID NO: 31 or 85.

63. An engineered 4-1BBL polypeptide comprising an amino acid sequence that is at least 80% identical to SEQ ID NO: 2, 32, 59, or 86, wherein the polypeptide comprises one or more of the following:
(a) the amino acid that corresponds to S62 of SEQ ID NO: 2 is T;
(b) the amino acid that corresponds to E107 of SEQ ID NO: 2 is M; and
(c) the amino acid that corresponds to A176 of SEQ ID NO: 2 is S.

64. The engineered 4-1BBL polypeptide of claim 63, comprising an amino acid sequence that is at least 90% identical to SEQ ID NO: 32 or 86.

65. An engineered 4-1BBL polypeptide comprising an amino acid sequence that is at least 80% identical to SEQ ID NO: 2, 33, 59, or 87, wherein the polypeptide comprises one or more of the following:
(a) the amino acid that corresponds to S62 of SEQ ID NO: 2 is A; and
(b) the amino acid that corresponds to E107 of SEQ ID NO: 2 is S.

66. The engineered 4-1BBL polypeptide of claim 65, comprising an amino acid sequence that is at least 90% identical to SEQ ID NO: 33 or 87.

67. An engineered 4-1BBL polypeptide comprising an amino acid sequence that is at least 80% identical to SEQ ID NO: 2, 34, 59, or 88, wherein the amino acid that corresponds to A176 of SEQ ID NO: 2 is Q.

68. The engineered 4-1BBL polypeptide of claim 67, comprising an amino acid sequence that is at least 90% identical to SEQ ID NO: 34 or 88.

69. An engineered 4-1BBL polypeptide comprising an amino acid sequence that is at least 80% identical to SEQ ID NO: 2, 35, 59, or 89, wherein the polypeptide comprises one or more of the following:
(a) the amino acid that corresponds to S62 of SEQ ID NO: 2 is P;
(b) the amino acid that corresponds to G106 of SEQ ID NO: 2 is F; and
(c) the amino acid that corresponds to A176 of SEQ ID NO: 2 is S.

70. The engineered 4-1BBL polypeptide of claim 69, comprising an amino acid sequence that is at least 90% identical to SEQ ID NO: 35 or 89.

71. An engineered 4-1BBL polypeptide comprising an amino acid sequence that is at least 80% identical to SEQ ID NO: 2, 36, 59, or 90, wherein the amino acid that corresponds to E107 of SEQ ID NO: 2 is S.

72. The engineered 4-1BBL polypeptide of claim 71, comprising an amino acid sequence that is at least 90% identical to SEQ ID NO: 36 or 90.

73. An engineered 4-1BBL polypeptide comprising an amino acid sequence that is at least 80% identical to SEQ ID NO: 2, 37, 59, or 91, wherein the polypeptide comprises one or more of the following:
(a) the amino acid that corresponds to G106 of SEQ ID NO: 2 is S; and
(b) the amino acid that corresponds to W177 of SEQ ID NO: 2 is F.

74. The engineered 4-1BBL polypeptide of claim 73, comprising an amino acid sequence that is at least 90% identical to SEQ ID NO: 37 or 91.

75. An engineered 4-1BBL polypeptide comprising an amino acid sequence that is at least 80% identical to SEQ ID NO: 2, 38, 59, or 92, wherein the polypeptide comprises one or more of the following:
(a) the amino acid that corresponds to P64 of SEQ ID NO: 2 is N; and
(b) the amino acid that corresponds to E107 of SEQ ID NO: 2 is I.

76. The engineered 4-1BBL polypeptide of claim 75, comprising an amino acid sequence that is at least 90% identical to SEQ ID NO: 38 or 92.

77. An engineered 4-1BBL polypeptide comprising an amino acid sequence that is at least 80% identical to SEQ ID NO: 2, 39, 59, or 93, wherein the polypeptide comprises one or more of the following:
(a) the amino acid that corresponds to S62 of SEQ ID NO: 2 is P; and
(b) the amino acid that corresponds to E107 of SEQ ID NO: 2 is A.

78. The engineered 4-1BBL polypeptide of claim 77, comprising an amino acid sequence that is at least 90% identical to SEQ ID NO: 39 or 93.

79. The engineered 4-1BBL polypeptide of any one of claims 1-78, wherein the engineered 4-1BBL polypeptide further comprises a CH2 domain and a CH3 domain.

80. The engineered 4-1BBL polypeptide of claim 79, wherein the engineered 4-1BBL polypeptide further comprises a hinge region.

81. The engineered 4-1BBL polypeptide of claim 79 or 80, wherein the CH2 domain is an IgG CH2 domain and the CH3 domain is an IgG CH3 domain.

82. The engineered 4-1BBL polypeptide of any one of claims 79-81, wherein the engineered 4-1BBL polypeptide comprises an amino acid sequence that is at least 80%, 85%, 90%, or 95% identical to any one of SEQ ID NOs: 41-58.

83. A protein construct comprising the engineered 4-1BBL polypeptide of any one of claims 1-82.

84. The protein construct of claim 83, comprising two or more engineered 4-1BBL polypeptides.

85. The protein construct of claim 84, wherein at least two engineered 4-1BBL polypeptides are identical.

86. The protein construct of claim 84, wherein at least two engineered 4-1BBL polypeptides are different.

87. The protein construct of claim 83, further comprising an Fc region.

88. The protein construct of claim 87, wherein the Fc region is an IgG4 Fc region.

89. The protein construct of claim 87, wherein the Fc region is an IgG1 Fc region (e.g., with LALA mutations or LALA-PG mutations).

90. The protein construct of any one of claims 87-89, wherein the engineered 4-1BBL polypeptide is connected to the C-terminus of the Fc region, optionally via a linker peptide.

91. The protein construct of any one of claims 87-89, wherein the engineered 4-1BBL polypeptide is connected to the N-terminus of the Fc region, optionally via a linker peptide.

92. A protein construct comprising
a first fusion polypeptide comprising the engineered 4-1BBL polypeptide of any one of claims 1-82, a first CH2 domain, and a first CH3 domain; and
a second fusion polypeptide comprising a second CH2 domain, and a second CH3 domain;
wherein the first fusion polypeptide and the second fusion polypeptide associate with each other, forming a dimer.

93. The protein construct of claim 92, wherein the second fusion polypeptide further comprises a second engineered 4-1BBL polypeptide.

94. A pharmaceutical composition comprising the engineered 4-1BBL polypeptide of any one of claims 1-82 or the protein construct of any one of claims 83-93; and
a pharmaceutically acceptable carrier.

95. A nucleic acid encoding the engineered 4-1BBL polypeptide of any one of claims 1-82 or the protein construct of any one of claims 83-93.

96. A vector comprising the nucleic acid of claim 95.

97. A cell comprising the nucleic acid of claim 95.

98. The cell of claim 97, wherein the cell is a CHO cell.

99. A method of producing an engineered 4-1BBL polypeptide or a protein construct comprising the engineered 4-1BBL polypeptide, the method comprising
(a) culturing the cell of claim 97 or 98 under conditions sufficient for the cell to produce the engineered 4-1BBL polypeptide or the protein construct; and
(b) collecting the engineered 4-1BBL polypeptide or the protein construct produced by the cell.

100. A method of treating a subject having cancer, the method comprising administering a therapeutically effective amount of a composition comprising the engineered 4-1BBL polypeptide of any one of claims 1-82 or the protein construct of any one of claims 83-93, to the subject.

101. The method of claim 100, wherein the subject has a solid tumor or a hematologic cancer.

102. The method of claim 100, wherein the cancer is breast cancer, oropharyngeal cancer, ovarian cancer, B cell lymphoma, or Non-Hodgkin's lymphoma, non-small cell lung cancer (NSCLC), melanoma, B-cell non-Hodgkin lymphoma, colorectal cancer, or multiple myeloma.

103. A method of decreasing the rate of tumor growth, the method comprising
contacting a tumor cell with an effective amount of a composition comprising the engineered 4-1BBL polypeptide of any one of claims 1-82 or the protein construct of any one of claims 83-93.

104. A method of killing a tumor cell, the method comprising
contacting a tumor cell with an effective amount of a composition comprising the engineered 4-1BBL polypeptide of any one of claims 1-82 or the protein construct of any one of claims 83-93.


SEQUENCE LISTING (104 sequences)

SEQ ID NO: 1 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVT

SEQ ID NO: 4 - protein, 347 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 5 aa, synthetic construct
GGGGS

SEQ ID NO: 6 - protein, 205 aa, synthetic construct
ACPWAVSGARASPGSAASPRLREGPELSPDDPAGLLDLRQGMFAQLVAQNVLLIDGPLSWYEDPGLAGVSLTGGLSYKEDTKELVVAKAGVYYVFFQLELRRVVAGTGSGSVSLALHLQPLRSAAGAAALALTVDLPPASSEARNSAFGFQGRLLHLSAGQRLGVHLHTEARARHALQLTQGATVLGLFRVTPEIPAGLPSPRSE

SEQ ID NO: 7 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLRQGATVLGLFRVTPEIPAGLPSPRSE

SEQ ID NO: 8 - protein, 205 aa, synthetic construct
ACPWAVSGARASPGSAASPRLREGPELSPDDPAGLLDLRQGMFAQLVAQNVLLIDGPLSWYTDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLSQGATVLGLFRVTPEIPAGLPSPRSE

SEQ ID NO: 9 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAQEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 10 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLAQGATVLGLFRVTPEIPAGLPSPRSE

SEQ ID NO: 11 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLEQGATVLGLFRVTPEIPAGLPSPRSE

SEQ ID NO: 12 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAKEGSGSVSLALHLQPLRSAAGAAALALTVDLPPASSEARNSAFGFQGRLLHLSAGQRLGVHLHTEARARHAWQLTQGQTVLGLFRVTPEIPAGLPSPRSE

SEQ ID NO: 13 - protein, 205 aa, synthetic construct
ACPWAVSGARASPGSAASPRLREGPELSPDDPAGLLDLRQGMFAQLVAQNVLLIDGPLSWYPDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 14 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGQGSGSVSLALHLQPLRSAAGAAALALTVDLPPASSEARNSAFGFQGRLLHLSAGQRLGVHLHTEARARHAWQLTQGRTVLGLFRVTPEIPAGLPSPRSE

SEQ ID NO: 15 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQVEFRRVVAGQGSGSVSLALHLQPLRSAAGAAALALTVDLPPASSEARNSAFGFQGRLLHLSAGQRLGVHLHTEARARHGWQLTQGATVLGLFRVTPEIPAGLPSPRSE

SEQ ID NO: 16 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAHEGSGSVSLALHLQPLRSAAGAAALALTVDLPPASSEARNSAFGFQGRLLHLSAGQRLGVHLHTEARARHAWQLAQGATVLGLFRVTPEIPAGLPSPRSE

SEQ ID NO: 17 - protein, 205 aa, synthetic construct
ACPWAVSGARASPGSAASPRLREGPELSPDDPAGLLDLRQGMFAQLVAQNVLLIDGPLSWYADPGLAGVSLTGGLSYKEDTKELVVAKAGVYYVFFQLELRRVVAGQGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 18 - protein, 205 aa, synthetic construct; identical to SEQ ID NO: 17

SEQ ID NO: 19 - protein, 205 aa, synthetic construct
ACPWAVSGARASPGSAASPRLREGPELSPDDPAGLLDLRQGMFAQLVAQNVLLIDGPLSWYNDPGLAGVSLTGGLSYKEDTKELVVAKAGVYYVFFQLELRRVVAGQGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 20 - protein, 205 aa, synthetic construct
ACPWAVSGARASPGSAASPRLREGPELSPDDPAGLLDLRQGMFAQLVAQNVLLIDGPLSWYTDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGRTVLGLFRVTPEIPAGLPSPRSE

SEQ ID NO: 21 - protein, 205 aa, synthetic construct
ACPWAVSGARASPGSAASPRLREGPELSPDDPAGLLDLRQGMFAQLVAQNVLLIDGPLSWYHDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 22 - protein, 205 aa, synthetic construct
ACPWAVSGARASPGSAASPRLREGPELSPDDPAGLLDLRQGMFAQLVAQNVLLIDGPLSWYPDPGLAGVSLTGGLSYKEDTKELVVAKAGVYYVFFQLELRRVVAGRGSGSVSLALHLQPLRSAAGAAALALTVDLPPASSEARNSAFGFQGRLLHLSAGQRLGVHLHTEARARHAWQFTQGATVLGLFRVTPEIPAGLPSPRSE

SEQ ID NO: 23 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGLGSGSVSLALHLQPLRSAAGAAALALTVDLPPASSEARNSAFGFQGRLLHLSAGQRLGVHLHTEARARHAMQLTQGATVLGLFRVTPEIPAGLPSPRSE

SEQ ID NO: 24 - protein, 205 aa, synthetic construct
ACPWAVSGARASPGSAASPRLREGPELSPDDPAGLLDLRQGMFAQLVAQNVLLIDGPLSWYPDPGLAGVSLTGGLSYKEDTKELVVAKAGVYYVFFQLELRRVVAREGSGSVSLALHLQPLRSAAGAAALALTVDLPPASSEARNSAFGFQGRLLHLSAGQRLGVHLHTEARARHAMQLTQGATVLGLFRVTPEIPAGLPSPRSE

SEQ ID NO: 25 - protein, 205 aa, synthetic construct
ACPWAVSGARASPGSAAIPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 26 - protein, 205 aa, synthetic construct
ACPWAVSGARASPGSAASPRLREGPELSPDDPAGLLDLRQGMFAQLVAQNVLLIDGPLSWYSDPGLAGVSLTGGLNYKEDTKELVVAKAGVYYVFFQLKLRRVVAGEGSSSVSLALHLQPLRSAAGAAALALTVDLPPASSEARNSAFGFQGRLLHLNAGQRLGVHLHTEARARHAWQLAQGATVLGLFRVTPEIPAGLPSPRSE

SEQ ID NO: 27 - protein, 205 aa, synthetic construct
ACPWAVSGARASPGSAASPRLREGPELSPDDPAGLLDLRQGMFAQLVAQNVLLIDGPLSWYADPGLAGVSLTGGLSYKEDTKELVVAKAGVYYVFFQLEVRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 28 - protein, 205 aa, synthetic construct
ACPWAVSGARASPGSAASPRLREGPELSPDDPAGLLDLRQGMFAQLVAQNVLLIDGPLSFYSDPGLAGVSLTGGLSYKEDTKELVVAKAGVYYVFFQLELRRVVAGTGSGSVSLALHLQPLRSAAGAAALALTVDLPPASSEARNSAFGFQGRLLHLSAGQRLGVHLHTEARARHAWQMTQGATVLGLFRVTPEIPAGLPSPRSE

SEQ ID NO: 29 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGQGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 30 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGKTVLGLFRVTPEIPAGLPSPRSE

SEQ ID NO: 31 - protein, 205 aa, synthetic construct
ACPWAVSGARASPGSAASPRLREGPELSPDDPAGLLDLRQGMFAQLVAQNVLLIDGPLSFYSDPGLAGVSLTGGLSYKEDTKELVVAKAGVYYVFFQLELRRVVAREGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 32 - protein, 205 aa, synthetic construct
ACPWAVSGARASPGSAASPRLREGPELSPDDPAGLLDLRQGMFAQLVAQNVLLIDGPLSWYTDPGLAGVSLTGGLSYKEDTKELVVAKAGVYYVFFQLELRRVVAGMGSGSVSLALHLQPLRSAAGAAALALTVDLPPASSEARNSAFGFQGRLLHLSAGQRLGVHLHTEARARHSWQLTQGATVLGLFRVTPEIPAGLPSPRSE

SEQ ID NO: 33 - protein, 205 aa, synthetic construct
ACPWAVSGARASPGSAASPRLREGPELSPDDPAGLLDLRQGMFAQLVAQNVLLIDGPLSWYADPGLAGVSLTGGLSYKEDTKELVVAKAGVYYVFFQLELRRVVAGSGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 34 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHQWQLTQGATVLGLFRVTPEIPAGLPSPRSE

SEQ ID NO: 35 - protein, 205 aa, synthetic construct
ACPWAVSGARASPGSAASPRLREGPELSPDDPAGLLDLRQGMFAQLVAQNVLLIDGPLSWYPDPGLAGVSLTGGLSYKEDTKELVVAKAGVYYVFFQLELRRVVAFEGSGSVSLALHLQPLRSAAGAAALALTVDLPPASSEARNSAFGFQGRLLHLSAGQRLGVHLHTEARARHSWQLTQGATVLGLFRVTPEIPAGLPSPRSE

SEQ ID NO: 36 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGSGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 37 - protein, 205 aa, synthetic construct
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVASEGSGSVSLALHLQPLRSAAGAAALALTVDLPPASSEARNSAFGFQGRLLHLSAGQRLGVHLHTEARARHAFQLTQGATVLGLFRVTPEIPAGLPSPRSE

SEQ ID NO: 38 - protein, 205 aa, synthetic construct
ACPWAVSGARASPGSAASPRLREGPELSPDDPAGLLDLRQGMFAQLVAQNVLLIDGPLSWYSDNGLAGVSLTGGLSYKEDTKELVVAKAGVYYVFFQLELRRVVAGIGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 39 - protein, 205 aa, synthetic construct
ACPWAVSGARASPGSAASPRLREGPELSPDDPAGLLDLRQGMFAQLVAQNVLLIDGPLSWYPDPGLAGVSLTGGLSYKEDTKELVVAKAGVYYVFFQLELRRVVAGAGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 40 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLRQGATVLGLFRVT

SEQ ID NO: 42 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDPGLAGVSLTGGLSYKEDTKELVVAKAGVYYVFFQLELRRVVAQEGSGSVSLALHLQPLRSAAGAAALALTVDLPPASSEARNSAFGFQGRLLHLSAGQRLGVHLHTEARARHAWQLTQGATVLGLFRVT

SEQ ID NO: 43 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYTDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLSQGATVLGLFRVT

SEQ ID NO: 44 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDPGLAGVSLTGGLSYKEDTKELVVAKAGVYYVFFQLELRRVVAKEGSGSVSLALHLQPLRSAAGAAALALTVDLPPASSEARNSAFGFQGRLLHLSAGQRLGVHLHTEARARHAWQLTQGQTVLGLFRVT

SEQ ID NO: 45 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDPGLAGVSLTGGLSYKEDTKELVVAKAGVYYVFFQLELRRVVAHEGSGSVSLALHLQPLRSAAGAAALALTVDLPPASSEARNSAFGFQGRLLHLSAGQRLGVHLHTEARARHAWQLAQGATVLGLFRVT

SEQ ID NO: 46 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDPGLAGVSLTGGLSYKEDTKELVVAKAGVYYVFFQLELRRVVAGAGSGSVSLALHLQPLRSAAGAAALALTVDLPPASSEARNSAFGFQGRLLHLSAGQRLGVHLHTEARARHAWQLTQGATVLGLFRVT

SEQ ID NO: 47 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYADPGLAGVSLTGGLSYKEDTKELVVAKAGVYYVFFQLELRRVVAGQGSGSVSLALHLQPLRSAAGAAALALTVDLPPASSEARNSAFGFQGRLLHLSAGQRLGVHLHTEARARHAWQLTQGATVLGLFRVT

SEQ ID NO: 48 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYNDPGLAGVSLTGGLSYKEDTKELVVAKAGVYYVFFQLELRRVVAGQGSGSVSLALHLQPLRSAAGAAALALTVDLPPASSEARNSAFGFQGRLLHLSAGQRLGVHLHTEARARHAWQLTQGATVLGLFRVT

SEQ ID NO: 49 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYTDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGRTVLGLFRVT

SEQ ID NO: 50 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDPGLAGVSLTGGLSYKEDTKELVVAKAGVYYVFFQLELRRVVAGLGSGSVSLALHLQPLRSAAGAAALALTVDLPPASSEARNSAFGFQGRLLHLSAGQRLGVHLHTEARARHAMQLTQGATVLGLFRVT

SEQ ID NO: 51 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLAQGATVLGLFRVT

SEQ ID NO: 52 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYADPGLAGVSLTGGLSYKEDTKELVVAKAGVYYVFFQLEVRRVVAGEGSGSVSLALHLQPLRSAAGAAALALTVDLPPASSEARNSAFGFQGRLLHLSAGQRLGVHLHTEARARHAWQLTQGATVLGLFRVT

SEQ ID NO: 53 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDPGLAGVSLTGGLSYKEDTKELVVAKAGVYYVFFQLELRRVVAGQGSGSVSLALHLQPLRSAAGAAALALTVDLPPASSEARNSAFGFQGRLLHLSAGQRLGVHLHTEARARHAWQLTQGATVLGLFRVT

SEQ ID NO: 54 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSFYSDPGLAGVSLTGGLSYKEDTKELVVAKAGVYYVFFQLELRRVVAREGSGSVSLALHLQPLRSAAGAAALALTVDLPPASSEARNSAFGFQGRLLHLSAGQRLGVHLHTEARARHAWQLTQGATVLGLFRVT

SEQ ID NO: 55 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYADPGLAGVSLTGGLSYKEDTKELVVAKAGVYYVFFQLELRRVVAGSGSGSVSLALHLQPLRSAAGAAALALTVDLPPASSEARNSAFGFQGRLLHLSAGQRLGVHLHTEARARHAWQLTQGATVLGLFRVT

SEQ ID NO: 56 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYTDPGLAGVSLTGGLSYKEDTKELVVAKAGVYYVFFQLELRRVVAGMGSGSVSLALHLQPLRSAAGAAALALTVDLPPASSEARNSAFGFQGRLLHLSAGQRLGVHLHTEARARHSWQLTQGATVLGLFRVT

SEQ ID NO: 57 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDPGLAGVSLTGGLSYKEDTKELVVAKAGVYYVFFQLELRRVVASEGSGSVSLALHLQPLRSAAGAAALALTVDLPPASSEARNSAFGFQGRLLHLSAGQRLGVHLHTEARARHAFQLTQGATVLGLFRVT

SEQ ID NO: 58 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDNGLAGVSLTGGLSYKEDTKELVVAKAGVYYVFFQLELRRVVAGIGSGSVSLALHLQPLRSAAGAAALALTVDLPPASSEARNSAFGFQGRLLHLSAGQRLGVHLHTEARARHAWQLTQGATVLGLFRVT

SEQ ID NO: 59 - protein, 152 aa, Homo sapiens
GMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVT

SEQ ID NO: 60 - protein, 152 aa, synthetic construct
GMFAQLVAQNVLLIDGPLSWYEDPGLAGVSLTGGLSYKEDTKELVVAKAGVYYVFFQLELRRVVAGTGSGSVSLALHLQPLRSAAGAAALALTVDLPPASSEARNSAFGFQGRLLHLSAGQRLGVHLHTEARARHALQLTQGATVLGLFRVT

SEQ ID NO: 61 - protein, 152 aa, synthetic construct
GMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLRQGATVLGLFRVT

SEQ ID NO: 62 - protein, 152 aa, synthetic construct
GMFAQLVAQNVLLIDGPLSWYTDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLSQGATVLGLFRVT

SEQ ID NO: 63 - protein, 152 aa, synthetic construct
GMFAQLVAQNVLLIDGPLSWYSDPGLAGVSLTGGLSYKEDTKELVVAKAGVYYVFFQLELRRVVAQEGSGSVSLALHLQPLRSAAGAAALALTVDLPPASSEARNSAFGFQGRLLHLSAGQRLGVHLHTEARARHAWQLTQGATVLGLFRVT

SEQ ID NO: 64 - protein, 152 aa, synthetic construct
GMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLAQGATVLGLFRVT

SEQ ID NO: 65 - protein, 152 aa, synthetic construct
GMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLEQGATVLGLFRVT

SEQ ID NO: 66 - protein, 152 aa, synthetic construct
GMFAQLVAQNVLLIDGPLSWYSDPGLAGVSLTGGLSYKEDTKELVVAKAGVYYVFFQLELRRVVAKEGSGSVSLALHLQPLRSAAGAAALALTVDLPPASSEARNSAFGFQGRLLHLSAGQRLGVHLHTEARARHAWQLTQGQTVLGLFRVT

SEQ ID NO: 67 - protein, 152 aa, synthetic construct
GMFAQLVAQNVLLIDGPLSWYPDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVT

SEQ ID NO: 68 - protein, 152 aa, synthetic construct
GMFAQLVAQNVLLIDGPLSWYSDPGLAGVSLTGGLSYKEDTKELVVAKAGVYYVFFQLELRRVVAGQGSGSVSLALHLQPLRSAAGAAALALTVDLPPASSEARNSAFGFQGRLLHLSAGQRLGVHLHTEARARHAWQLTQGRTVLGLFRVT

SEQ ID NO: 69 - protein, 152 aa, synthetic construct
GMFAQLVAQNVLLIDGPLSWYSDPGLAGVSLTGGLSYKEDTKELVVAKAGVYYVFFQVEFRRVVAGQGSGSVSLALHLQPLRSAAGAAALALTVDLPPASSEARNSAFGFQGRLLHLSAGQRLGVHLHTEARARHGWQLTQGATVLGLFRVT

SEQ ID NO: 70 - protein, 152 aa, synthetic construct
GMFAQLVAQNVLLIDGPLSWYSDPGLAGVSLTGGLSYKEDTKELVVAKAGVYYVFFQLELRRVVAHEGSGSVSLALHLQPLRSAAGAAALALTVDLPPASSEARNSAFGFQGRLLHLSAGQRLGVHLHTEARARHAWQLAQGATVLGLFRVT

SEQ ID NO: 71 - protein, 152 aa, synthetic construct
GMFAQLVAQNVLLIDGPLSWYSDPGLAGVSLTGGLSYKEDTKELVVAKAGVYYVFFQLELRRVVAGAGSGSVSLALHLQPLRSAAGAAALALTVDLPPASSEARNSAFGFQGRLLHLSAGQRLGVHLHTEARARHAWQLTQGATVLGLFRVT

SEQ ID NO: 72 - protein, 152 aa, synthetic construct
GMFAQLVAQNVLLIDGPLSWYADPGLAGVSLTGGLSYKEDTKELVVAKAGVYYVFFQLELRRVVAGQGSGSVSLALHLQPLRSAAGAAALALTVDLPPASSEARNSAFGFQGRLLHLSAGQRLGVHLHTEARARHAWQLTQGATVLGLFRVT

SEQ ID NO: 73 - protein, 152 aa, synthetic construct
GMFAQLVAQNVLLIDGPLSWYNDPGLAGVSLTGGLSYKEDTKELVVAKAGVYYVFFQLELRRVVAGQGSGSVSLALHLQPLRSAAGAAALALTVDLPPASSEARNSAFGFQGRLLHLSAGQRLGVHLHTEARARHAWQLTQGATVLGLFRVT

SEQ ID NO: 74 - protein, 152 aa, synthetic construct
GMFAQLVAQNVLLIDGPLSWYTDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGRTVLGLFRVT

SEQ ID NO: 75 - protein, 152 aa, synthetic construct
GMFAQLVAQNVLLIDGPLSWYHDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVT

SEQ ID NO: 76 - protein, 152 aa, synthetic construct
GMFAQLVAQNVLLIDGPLSWYPDPGLAGVSLTGGLSYKEDTKELVVAKAGVYYVFFQLELRRVVAGRGSGSVSLALHLQPLRSAAGAAALALTVDLPPASSEARNSAFGFQGRLLHLSAGQRLGVHLHTEARARHAWQFTQGATVLGLFRVT

SEQ ID NO: 77 - protein, 152 aa, synthetic construct
GMFAQLVAQNVLLIDGPLSWYSDPGLAGVSLTGGLSYKEDTKELVVAKAGVYYVFFQLELRRVVAGLGSGSVSLALHLQPLRSAAGAAALALTVDLPPASSEARNSAFGFQGRLLHLSAGQRLGVHLHTEARARHAMQLTQGATVLGLFRVT

SEQ ID NO: 78 - protein, 152 aa, synthetic construct
GMFAQLVAQNVLLIDGPLSWYPDPGLAGVSLTGGLSYKEDTKELVVAKAGVYYVFFQLELRRVVAREGSGSVSLALHLQPLRSAAGAAALALTVDLPPASSEARNSAFGFQGRLLHLSAGQRLGVHLHTEARARHAMQLTQGATVLGLFRVT

SEQ ID NO: 79 - protein, 152 aa, synthetic construct; identical to SEQ ID NO: 59

SEQ ID NO: 80 - protein, 152 aa, synthetic construct
GMFAQLVAQNVLLIDGPLSWYSDPGLAGVSLTGGLNYKEDTKELVVAKAGVYYVFFQLKLRRVVAGEGSSSVSLALHLQPLRSAAGAAALALTVDLPPASSEARNSAFGFQGRLLHLNAGQRLGVHLHTEARARHAWQLAQGATVLGLFRVT

SEQ ID NO: 81 - protein, 152 aa, synthetic construct
GMFAQLVAQNVLLIDGPLSWYADPGLAGVSLTGGLSYKEDTKELVVAKAGVYYVFFQLEVRRVVAGEGSGSVSLALHLQPLRSAAGAAALALTVDLPPASSEARNSAFGFQGRLLHLSAGQRLGVHLHTEARARHAWQLTQGATVLGLFRVT

SEQ ID NO: 82 - protein, 152 aa, synthetic construct
GMFAQLVAQNVLLIDGPLSFYSDPGLAGVSLTGGLSYKEDTKELVVAKAGVYYVFFQLELRRVVAGTGSGSVSLALHLQPLRSAAGAAALALTVDLPPASSEARNSAFGFQGRLLHLSAGQRLGVHLHTEARARHAWQMTQGATVLGLFRVT

SEQ ID NO: 83 - protein, 152 aa, synthetic construct
GMFAQLVAQNVLLIDGPLSWYSDPGLAGVSLTGGLSYKEDTKELVVAKAGVYYVFFQLELRRVVAGQGSGSVSLALHLQPLRSAAGAAALALTVDLPPASSEARNSAFGFQGRLLHLSAGQRLGVHLHTEARARHAWQLTQGATVLGLFRVT

SEQ ID NO: 84 - protein, 152 aa, synthetic construct
GMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGKTVLGLFRVT

SEQ ID NO: 85 - protein, 152 aa, synthetic construct
GMFAQLVAQNVLLIDGPLSFYSDPGLAGVSLTGGLSYKEDTKELVVAKAGVYYVFFQLELRRVVAREGSGSVSLALHLQPLRSAAGAAALALTVDLPPASSEARNSAFGFQGRLLHLSAGQRLGVHLHTEARARHAWQLTQGATVLGLFRVT

SEQ ID NO: 86 - protein, 152 aa, synthetic construct
GMFAQLVAQNVLLIDGPLSWYTDPGLAGVSLTGGLSYKEDTKELVVAKAGVYYVFFQLELRRVVAGMGSGSVSLALHLQPLRSAAGAAALALTVDLPPASSEARNSAFGFQGRLLHLSAGQRLGVHLHTEARARHSWQLTQGATVLGLFRVT

SEQ ID NO: 87 - protein, 152 aa, synthetic construct
GMFAQLVAQNVLLIDGPLSWYADPGLAGVSLTGGLSYKEDTKELVVAKAGVYYVFFQLELRRVVAGSGSGSVSLALHLQPLRSAAGAAALALTVDLPPASSEARNSAFGFQGRLLHLSAGQRLGVHLHTEARARHAWQLTQGATVLGLFRVT

SEQ ID NO: 88 - protein, 152 aa, synthetic construct
GMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHQWQLTQGATVLGLFRVT

SEQ ID NO: 89 - protein, 152 aa, synthetic construct
GMFAQLVAQNVLLIDGPLSWYPDPGLAGVSLTGGLSYKEDTKELVVAKAGVYYVFFQLELRRVVAFEGSGSVSLALHLQPLRSAAGAAALALTVDLPPASSEARNSAFGFQGRLLHLSAGQRLGVHLHTEARARHSWQLTQGATVLGLFRVT

SEQ ID NO: 90 - protein, 152 aa, synthetic construct
GMFAQLVAQNVLLIDGPLSWYSDPGLAGVSLTGGLSYKEDTKELVVAKAGVYYVFFQLELRRVVAGSGSGSVSLALHLQPLRSAAGAAALALTVDLPPASSEARNSAFGFQGRLLHLSAGQRLGVHLHTEARARHAWQLTQGATVLGLFRVT

SEQ ID NO: 91 - protein, 152 aa, synthetic construct
GMFAQLVAQNVLLIDGPLSWYSDPGLAGVSLTGGLSYKEDTKELVVAKAGVYYVFFQLELRRVVASEGSGSVSLALHLQPLRSAAGAAALALTVDLPPASSEARNSAFGFQGRLLHLSAGQRLGVHLHTEARARHAFQLTQGATVLGLFRVT

SEQ ID NO: 92 - protein, 152 aa, synthetic construct
GMFAQLVAQNVLLIDGPLSWYSDNGLAGVSLTGGLSYKEDTKELVVAKAGVYYVFFQLELRRVVAGIGSGSVSLALHLQPLRSAAGAAALALTVDLPPASSEARNSAFGFQGRLLHLSAGQRLGVHLHTEARARHAWQLTQGATVLGLFRVT

SEQ ID NO: 93 - protein, 152 aa, synthetic construct
GMFAQLVAQNVLLIDGPLSWYPDPGLAGVSLTGGLSYKEDTKELVVAKAGVYYVFFQLELRRVVAGAGSGSVSLALHLQPLRSAAGAAALALTVDLPPASSEARNSAFGFQGRLLHLSAGQRLGVHLHTEARARHAWQLTQGATVLGLFRVT

SEQ ID NO: 94 - protein, 191 aa, Homo sapiens
SAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 95 - protein, 26 aa, synthetic construct
TSGPGGQHHHHHHGAEQKLISEEDLS

SEQ ID NO: 96 - protein, 13 aa, synthetic construct
TSGPGGQHHHHHH

SEQ ID NO: 97 - protein, 26 aa, synthetic construct
TSGPGGQHHHHHHGAHKKLISEEDLS

SEQ ID NO: 98 - protein, 12 aa, synthetic construct
SGPGGQHHHHHH

SEQ ID NO: 99 - protein, 12 aa, synthetic construct
GGGGSGGGGSRQ

SEQ ID NO: 100 - protein, 10 aa, synthetic construct
GGGGSGGGGS

SEQ ID NO: 101 - protein, 441 aa, synthetic construct
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 102 - protein, 214 aa, synthetic construct
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 103 - protein, 447 aa, synthetic construct
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 104 - protein, 216 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC